SEQUENCE LISTING (22 sequences)

SEQ ID NO: 1 - DNA, 5775 bp, synthetic construct
  source 1..5775: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaaatgcggtgtgaaataccgcacagatgcgtaaggagaaaataccgcatcaggcgccattcgccattcaggctgcgcaactgttgggaagggcgatcggtgcgggcctcatcgctattacgccagctggcgaaagggggatgtgctgcaaggcgattaagttgggtaacgccagggttttcccagtcacgacgttgtaaaacgacggccagtgcaacgcgatgacgatggatagcgattcatcgatgagctgacccgatcgccgccgccggagggttgcgtttgagacgggcgacagatgtcgtaccgactggtagatgacagcaaacctgcccacatcgtcgctgaacgaattccgttcatacctattttgtcacttgagcatcgactttgggttccgtcgttgcccgttttgccaatgatcaacttgcatgctgttctgtctgtcgcacttgttgctttatgatgaagtgtcttagcacaatgttaggatatacatggcattcaaatgaatttgcgcgcgtgtggcatcccagctgggtactgtgtgcgtcgtctggctgagttgggagtcttgtgacggcgaccggtcctctgccgaaaatttaacgcggcggcttctaaatctctaccgctgcactgctgagtttgattgttcgtcaatcccgtcgttgcgaacgcttgaccgcgaaaaatttgggcatcgttagcggatgaatcagctgagacgtcgcacgtggtcagcgccgctcgttcattcgcgtgcgctgaactatcgataaagaaattcttggacgcggctgagaaatccaactgtacggtggacattccaccgctggtgcttgttaaatcatccctcttgatatcgaacttattacattgacagacacgcgcaacgatatccccccagtatggtgcccgcaattgcgtccttaacgttaaatgcgcccgcgtttctcgggtttcccattttcttttcgccttttgtggcttaagtatggtttgcttgataacgcatctctgtcgagttcccaccctatcttcggtcaccgtgatgttcgtccggtaaacatgagcctttacccgggctgactgttctcataacacggttcccgtgcccattaaattattgggaccttatttgtacaaacatcgtcattaggtcgcttcgctccccagttgatgcgtgttttgtgtcgctccgaaagcgatatgcccgcgaacccaatttgaacgttaggaaaggggtgaaccgtcttgtcggtggggatctttccggtattggccttaatcttgagctacggacccaagatcgtgcgcacgacgtgaggtccgtgaaacacgtgctaataaggcttctgcggcacaggaaacgtcgcagatactcttacgtaacgtaccgcaatttcttttagcctttatttacgtttattggtctcctcttttatcaaatttgcatacgtattcatcttaccagaatgatttggagtcaaaatgatgcatggacattgcccccattcagcacaaatttgagtaggagtctccttggtagtcttacaccctatttccaatttattggaccgcccccgtacctctcgtgtttgataatccgccttattgtaacttacagttacaattccctataagttccaagcgattatgaatcgtaaggctcttctggatagctggcctgtgccccctgccgtggtatctcaactgactagtccttggattaaggctatcttggggtcggagttctactcagatactacccagaacaccgccattcgctcttcgtattggaaaacgcaacacattcgttcttggccattacctgccgtagttatcggtcgttgggcggttcaaaatttagggttggcgattgcgatggtgaaggttggaacagatctggttgttggtcgctgtggggacgattccacaagtcgtaagaagaatctgatttgactgaaacttaatcagatgctttggtccacgactaccatctgagcggtcgggtaatgcctgagtaccgatatggtaatcaagtttgacaatgcagtcacgctattcctttatccattttaacatggtgagcgcttagagaccagttactccgagtgcagctcccgcgtacatatctacagccgttcggcggaaatgtaatctcgtcattaaggtcgcttgccccttattttaagccgtgtgcgtcgtatgaatcgtctgtgatcagagggcgtcgtcatgcgcgacgcggttttcatttttttcacctgcattcacatttgttcgccgtgtcgcaccgaccgtaccagtacgttacctcgtgtctatctggtgggttactacgttgatgctcttatgtgcgggcaggttatgtaggttgagagatcgttgacggctagctcagtcctaggtacagtgctagctactagtgaaagaggagaaatactagatgtcgaagggcgaagagcttttcacaggggtagtaccaattttagtagagttggatggtgatgtaaatggtcataagttttcagtacgtggggaaggtgaaggggacgcgacaaatggtaaattgactcttaaatttatctgcactaccggcaaattacccgttccatggcctactctggtgacgacactgacctacggtgttcagtgtttcagtcgttaccctgaccatatgaaacgccatgatttcttcaagtcggctatgcccgaaggctacgttcaggaacgtacaatttctttcaaggatgacgggacgtacaagactcgtgctgaagtaaagtttgaaggggatacattggtaaatcgtattgagttgaagggtattgactttaaggaggatggaaacatcttaggccataagcttgaatacaacttcaactcccacaacgtttatatcacagctgacaaacagaaaaacggaattaaagctaactttaagatccgccacaatgttgaagacggaagcgttcaattagcggatcactaccagcagaacacacccattggggatggacctgtcttgttgccagataatcattatctgagtacacaatccgtgttatcaaaggacccgaatgaaaagcgtgaccacatggtgctgcttgagtttgtcacagcggcagggatcacgcacggcatggacgaactgtacaagtaaaacggcccggagggtggcgggcaggacgcccgccataaactgccaggcatcaaattaagcagaaggccatcctgacggatggcctttttgcgtttgcaccagtgatcagggttattcccttgggagatccgaaaacgggcgtatagtcgagaccatcagttctggaccagcgagctgtgctgcgactcgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgtcgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactctacctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - DNA, 809 bp, synthetic construct
  source 1..809: mol_type = other DNA
ggagggttgcgtttgagacgggcgacagatgtcgtaccgactggtagatgacagcaaacctgcccacatcgtcgctgaacgaattccgttcatacctattttgtcacttgagcatcgactttgggttccgtcgttgcccgttttgccaatgatcaacttgcatgctgttctgtctgtcgcacttgttgctttatgatgaagtgtcttagcacaatgttaggatatacatggcattcaaatgaatttgcgcgcgtgtggcatcccagctgggtactgtgtgcgtcgtctggctgagttgggagtcttgtgacggcgaccggtcctctgccgaaaatttaacgcggcggcttctaaatctctaccgctgcactgctgagtttgattgttcgtcaatcccgtcgttgcgaacgcttgaccgcgaaaaatttgggcatcgttagcggatgaatcagctgagacgtcgcacgtggtcagcgccgctcgttcattcgcgtgcgctgaactatcgataaagaaattcttggacgcggctgagaaatccaactgtacggtggacattccaccgctggtgcttgttaaatcatccctcttgatatcgaacttattacattgacagacacgcgcaacgatatccccccagtatggtgcccgcaattgcgtccttaacgttaaatgcgcccgcgtttctcgggtttcccattttcttttcgccttttgtggcttaagtatggtttgcttgataacgcatctctgtcgagttcccaccctatcttcggtcaccgtgatgttcgtccggtaaacatgagcct

SEQ ID NO: 3 - DNA, 864 bp, synthetic construct
  source 1..864: mol_type = other DNA
ccgtgatgttcgtccggtaaacatgagcctttacccgggctgactgttctcataacacggttcccgtgcccattaaattattgggaccttatttgtacaaacatcgtcattaggtcgcttcgctccccagttgatgcgtgttttgtgtcgctccgaaagcgatatgcccgcgaacccaatttgaacgttaggaaaggggtgaaccgtcttgtcggtggggatctttccggtattggccttaatcttgagctacggacccaagatcgtgcgcacgacgtgaggtccgtgaaacacgtgctaataaggcttctgcggcacaggaaacgtcgcagatactcttacgtaacgtaccgcaatttcttttagcctttatttacgtttattggtctcctcttttatcaaatttgcatacgtattcatcttaccagaatgatttggagtcaaaatgatgcatggacattgcccccattcagcacaaatttgagtaggagtctccttggtagtcttacaccctatttccaatttattggaccgcccccgtacctctcgtgtttgataatccgccttattgtaacttacagttacaattccctataagttccaagcgattatgaatcgtaaggctcttctggatagctggcctgtgccccctgccgtggtatctcaactgactagtccttggattaaggctatcttggggtcggagttctactcagatactacccagaacaccgccattcgctcttcgtattggaaaacgcaacacattcgttcttggccattacctgccgtagttatcggtcgttgggcggttcaaaatttagggttggcgattgcgatggtgaaggttggaacagatctggt

SEQ ID NO: 4 - DNA, 788 bp, synthetic construct
  source 1..788: mol_type = other DNA
tgcgatggtgaaggttggaacagatctggttgttggtcgctgtggggacgattccacaagtcgtaagaagaatctgatttgactgaaacttaatcagatgctttggtccacgactaccatctgagcggtcgggtaatgcctgagtaccgatatggtaatcaagtttgacaatgcagtcacgctattcctttatccattttaacatggtgagcgcttagagaccagttactccgagtgcagctcccgcgtacatatctacagccgttcggcggaaatgtaatctcgtcattaaggtcgcttgccccttattttaagccgtgtgcgtcgtatgaatcgtctgtgatcagagggcgtcgtcatgcgcgacgcggttttcatttttttcacctgcattcacatttgttcgccgtgtcgcaccgaccgtaccagtacgttacctcgtgtctatctggtgggttactacgttgatgctcttatgtgcgggcaggttatgtaggttgagagatcgttgacggctagctcagtcctaggtacagtgctagctactagtgaaagaggagaaatactagatgtcgaagggcgaagagcttttcacaggggtagtaccaattttagtagagttggatggtgatgtaaatggtcataagttttcagtacgtggggaaggtgaaggggacgcgacaaatggtaaattgactcttaaatttatctgcactaccggcaaattacccgttccatggcctactctggtgacgacactgacctacggtgttcagtgtttcagtcgt

SEQ ID NO: 5 - DNA, 788 bp, synthetic construct; identical to SEQ ID NO: 4
  source 1..788: mol_type = other DNA

SEQ ID NO: 6 - DNA, 2752 bp, synthetic construct
  source 1..2752: mol_type = other DNA
atcagttctggaccagcgagctgtgctgcgactcgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgtcgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactctacctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaaatgcggtgtgaaataccgcacagatgcgtaaggagaaaataccgcatcaggcgccattcgccattcaggctgcgcaactgttgggaagggcgatcggtgcgggcctcatcgctattacgccagctggcgaaagggggatgtgctgcaaggcgattaagttgggtaacgccagggttttcccagtcacgacgttgtaaaacgacggccagtgcaacgcgatgacgatggatagcgattcatcgatgagctgacccgatcgccgccgccggagggttgcgtttgagacgggcgacagat

SEQ ID NO: 7 - DNA, 4718 bp, synthetic construct
  source 1..4718: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaaatgcggtgtgaaataccgcacagatgcgtaaggagaaaataccgcatcaggcgccattcgccattcaggctgcgcaactgttgggaagggcgatcggtgcgggcctcatcgctattacgccagctggcgaaagggggatgtgctgcaaggcgattaagttgggtaacgccagggttttcccagtcacgacgttgtaaaacgacggccagtgcaacgcgatgacgatggatagcgattcatcgatgagctgacccgatcgccgccgccggagggttgcgtttgagacgggcgacagataggccataaattgggtgctgaagtctttgcgagagattgtcttgattttggttgggaagatttagcttctacaattaataacatgatgtatcttgatataccgtttattgcttttactgcaaataacgataattgggtcaagcaagatgaagttatcacattgttatcaaatattcgtagtaatcgatgcaagatatattctttgttaggaagttcgcatgacttgagtgaaaatttagtggtcctgcgcaatttttatcaatcggttacgaaagccgctatcgcgatggataatgatcatctggatattgatgttgatattactgaaccgtcatttgaacatttaactattgcgacagtcaatgaacgccgaatgagaattgagattgaaaatcaagcaatttctctgtcttaatgcgggcaggttatgtaggttgagagatcgttgacggctagctcagtcctaggtacagtgctagctactagtgaaagaggagaaatactagatgtcgaagggcgaagagcttttcacaggggtagtaccaattttagtagagttggatggtgatgtaaatggtcataagttttcagtacgtggggaaggtgaaggggacgcgacaaatggtaaattgactcttaaatttatctgcactaccggcaaattacccgttccatggcctactctggtgacgacactgacctacggtgttcagtgtttcagtcgttaccctgaccatatgaaacgccatgatttcttcaagtcggctatgcccgaaggctacgttcaggaacgtacaatttctttcaaggatgacgggacgtacaagactcgtgctgaagtaaagtttgaaggggatacattggtaaatcgtattgagttgaagggtattgactttaaggaggatggaaacatcttaggccataagcttgaatacaacttcaactcccacaacgtttatatcacagctgacaaacagaaaaacggaattaaagctaactttaagatccgccacaatgttgaagacggaagcgttcaattagcggatcactaccagcagaacacacccattggggatggacctgtcttgttgccagataatcattatctgagtacacaatccgtgttatcaaaggacccgaatgaaaagcgtgaccacatggtgctgcttgagtttgtcacagcggcagggatcacgcacggcatggacgaactgtacaagtaaaacggcccggagggtggcgggcaggacgcccgccataaactgccaggcatcaaattaagcagaaggccatcctgacggatggcctttttgcgtttgcaccagtgatcagggttattcccttgggaaatctattgagatattctatcactcaaatagcaatataaggactctctgaattcatgaaatttggaaactttttacttacataccaacctccccaattttctcaaacagaggtaatggaacgtttggttaaattaggtcgcatctctgaggagtgtggttttgataccgtatggttactggagcatcatttcacggagtttggtctacttggtaacccttatgtcgctgctgcatatttacttggcgcgactaaaaaattgaatgtaggaaccgccgctattgttcttcccacagcccatccagtacgccaacttgaagatgtgaatttattggatcaaatgtcaaaaggacgatttcggtttggtatttgccgagggctttacaacaaggactttcgcgtattcggcgcggatatgaataacagtcgcgccttagcggaatgctggtacgggctgataaagaatggcatgacagagggatatatggaagctgataatgaacatatcaagttccataaggtaaaagtaaaccccgcggcgtatagcagaggtggcgcaccggtttatgtggtggctgaatcagctgcgacgactgagtgggcagctcaatttggcctaatcagttctggaccagcgagctgtgctgcgactcgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgtcgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactctacctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 8 - DNA, 713 bp, synthetic construct
  source 1..713: mol_type = other DNA
ccgccggagggttgcgtttgagacgggcgacagataggccataaattgggtgctgaagtctttgcgagagattgtcttgattttggttgggaagatttagcttctacaattaataacatgatgtatcttgatataccgtttattgcttttactgcaaataacgataattgggtcaagcaagatgaagttatcacattgttatcaaatattcgtagtaatcgatgcaagatatattctttgttaggaagttcgcatgacttgagtgaaaatttagtggtcctgcgcaatttttatcaatcggttacgaaagccgctatcgcgatggataatgatcatctggatattgatgttgatattactgaaccgtcatttgaacatttaactattgcgacagtcaatgaacgccgaatgagaattgagattgaaaatcaagcaatttctctgtcttaatgcgggcaggttatgtaggttgagagatcgttgacggctagctcagtcctaggtacagtgctagctactagtgaaagaggagaaatactagatgtcgaagggcgaagagcttttcacaggggtagtaccaattttagtagagttggatggtgatgtaaatggtcataagttttcagtacgtggggaaggtgaaggggacgcgacaaatggtaaattgactcttaaatttatctgcactaccggcaaattacccgttccatggc

SEQ ID NO: 9 - DNA, 658 bp, synthetic construct
  source 1..658: mol_type = other DNA
tctgcactaccggcaaattacccgttccatggcctactctggtgacgacactgacctacggtgttcagtgtttcagtcgttaccctgaccatatgaaacgccatgatttcttcaagtcggctatgcccgaaggctacgttcaggaacgtacaatttctttcaaggatgacgggacgtacaagactcgtgctgaagtaaagtttgaaggggatacattggtaaatcgtattgagttgaagggtattgactttaaggaggatggaaacatcttaggccataagcttgaatacaacttcaactcccacaacgtttatatcacagctgacaaacagaaaaacggaattaaagctaactttaagatccgccacaatgttgaagacggaagcgttcaattagcggatcactaccagcagaacacacccattggggatggacctgtcttgttgccagataatcattatctgagtacacaatccgtgttatcaaaggacccgaatgaaaagcgtgaccacatggtgctgcttgagtttgtcacagcggcagggatcacgcacggcatggacgaactgtacaagtaaaacggcccggagggtggcgggcaggacgcccgccataaactgccaggcatcaaattaagcagaaggccatcctgacggat

SEQ ID NO: 10 - DNA, 732 bp, synthetic construct
  source 1..732: mol_type = other DNA
gcatcaaattaagcagaaggccatcctgacggatggcctttttgcgtttgcaccagtgatcagggttattcccttgggaaatctattgagatattctatcactcaaatagcaatataaggactctctgaattcatgaaatttggaaactttttacttacataccaacctccccaattttctcaaacagaggtaatggaacgtttggttaaattaggtcgcatctctgaggagtgtggttttgataccgtatggttactggagcatcatttcacggagtttggtctacttggtaacccttatgtcgctgctgcatatttacttggcgcgactaaaaaattgaatgtaggaaccgccgctattgttcttcccacagcccatccagtacgccaacttgaagatgtgaatttattggatcaaatgtcaaaaggacgatttcggtttggtatttgccgagggctttacaacaaggactttcgcgtattcggcgcggatatgaataacagtcgcgccttagcggaatgctggtacgggctgataaagaatggcatgacagagggatatatggaagctgataatgaacatatcaagttccataaggtaaaagtaaaccccgcggcgtatagcagaggtggcgcaccggtttatgtggtggctgaatcagctgcgacgactgagtgggcagctcaatttggcctaatcagttctggaccagcgagctgtgctgcgactcg

SEQ ID NO: 11 - DNA, 705 bp, synthetic construct
  source 1..705: mol_type = other DNA
ggagggttgcgtttgagacgggcgacagataggccataaattgggtgctgaagtctttgcgagagattgtcttgattttggttgggaagatttagcttctacaattaataacatgatgtatcttgatataccgtttattgcttttactgcaaataacgataattgggtcaagcaagatgaagttatcacattgttatcaaatattcgtagtaatcgatgcaagatatattctttgttaggaagttcgcatgacttgagtgaaaatttagtggtcctgcgcaatttttatcaatcggttacgaaagccgctatcgcgatggataatgatcatctggatattgatgttgatattactgaaccgtcatttgaacatttaactattgcgacagtcaatgaacgccgaatgagaattgagattgaaaatcaagcaatttctctgtcttaatgcgggcaggttatgtaggttgagagatcgttgacggctagctcagtcctaggtacagtgctagctactagtgaaagaggagaaatactagatgtcgaagggcgaagagcttttcacaggggtagtaccaattttagtagagttggatggtgatgtaaatggtcataagttttcagtacgtggggaaggtgaaggggacgcgacaaatggtaaattgactcttaaatttatctgcactaccggcaaattacccgttccat

SEQ ID NO: 12 - DNA, 654 bp, synthetic construct
  source 1..654: mol_type = other DNA
tctgcactaccggcaaattacccgttccatggcctactctggtgacgacactgacctacggtgttcagtgtttcagtcgttaccctgaccatatgaaacgccatgatttcttcaagtcggctatgcccgaaggctacgttcaggaacgtacaatttctttcaaggatgacgggacgtacaagactcgtgctgaagtaaagtttgaaggggatacattggtaaatcgtattgagttgaagggtattgactttaaggaggatggaaacatcttaggccataagcttgaatacaacttcaactcccacaacgtttatatcacagctgacaaacagaaaaacggaattaaagctaactttaagatccgccacaatgttgaagacggaagcgttcaattagcggatcactaccagcagaacacacccattggggatggacctgtcttgttgccagataatcattatctgagtacacaatccgtgttatcaaaggacccgaatgaaaagcgtgaccacatggtgctgcttgagtttgtcacagcggcagggatcacgcacggcatggacgaactgtacaagtaaaacggcccggagggtggcgggcaggacgcccgccataaactgccaggcatcaaattaagcagaaggccatcctgac

SEQ ID NO: 13 - DNA, 727 bp, synthetic construct
  source 1..727: mol_type = other DNA
gcatcaaattaagcagaaggccatcctgacggatggcctttttgcgtttgcaccagtgatcagggttattcccttgggaaatctattgagatattctatcactcaaatagcaatataaggactctctgaattcatgaaatttggaaactttttacttacataccaacctccccaattttctcaaacagaggtaatggaacgtttggttaaattaggtcgcatctctgaggagtgtggttttgataccgtatggttactggagcatcatttcacggagtttggtctacttggtaacccttatgtcgctgctgcatatttacttggcgcgactaaaaaattgaatgtaggaaccgccgctattgttcttcccacagcccatccagtacgccaacttgaagatgtgaatttattggatcaaatgtcaaaaggacgatttcggtttggtatttgccgagggctttacaacaaggactttcgcgtattcggcgcggatatgaataacagtcgcgccttagcggaatgctggtacgggctgataaagaatggcatgacagagggatatatggaagctgataatgaacatatcaagttccataaggtaaaagtaaaccccgcggcgtatagcagaggtggcgcaccggtttatgtggtggctgaatcagctgcgacgactgagtgggcagctcaatttggcctaatcagttctggaccagcgagctgtgctgcg

SEQ ID NO: 14 - DNA, 698 bp, synthetic construct
  source 1..698: mol_type = other DNA
gttgcgtttgagacgggcgacagataggccataaattgggtgctgaagtctttgcgagagattgtcttgattttggttgggaagatttagcttctacaattaataacatgatgtatcttgatataccgtttattgcttttactgcaaataacgataattgggtcaagcaagatgaagttatcacattgttatcaaatattcgtagtaatcgatgcaagatatattctttgttaggaagttcgcatgacttgagtgaaaatttagtggtcctgcgcaatttttatcaatcggttacgaaagccgctatcgcgatggataatgatcatctggatattgatgttgatattactgaaccgtcatttgaacatttaactattgcgacagtcaatgaacgccgaatgagaattgagattgaaaatcaagcaatttctctgtcttaatgcgggcaggttatgtaggttgagagatcgttgacggctagctcagtcctaggtacagtgctagctactagtgaaagaggagaaatactagatgtcgaagggcgaagagcttttcacaggggtagtaccaattttagtagagttggatggtgatgtaaatggtcataagttttcagtacgtggggaaggtgaaggggacgcgacaaatggtaaattgactcttaaatttatctgcactaccggcaaattacccgttcc

SEQ ID NO: 15 - DNA, 646 bp, synthetic construct
  source 1..646: mol_type = other DNA
gcactaccggcaaattacccgttccatggcctactctggtgacgacactgacctacggtgttcagtgtttcagtcgttaccctgaccatatgaaacgccatgatttcttcaagtcggctatgcccgaaggctacgttcaggaacgtacaatttctttcaaggatgacgggacgtacaagactcgtgctgaagtaaagtttgaaggggatacattggtaaatcgtattgagttgaagggtattgactttaaggaggatggaaacatcttaggccataagcttgaatacaacttcaactcccacaacgtttatatcacagctgacaaacagaaaaacggaattaaagctaactttaagatccgccacaatgttgaagacggaagcgttcaattagcggatcactaccagcagaacacacccattggggatggacctgtcttgttgccagataatcattatctgagtacacaatccgtgttatcaaaggacccgaatgaaaagcgtgaccacatggtgctgcttgagtttgtcacagcggcagggatcacgcacggcatggacgaactgtacaagtaaaacggcccggagggtggcgggcaggacgcccgccataaactgccaggcatcaaattaagcagaaggccatc

SEQ ID NO: 16 - DNA, 722 bp, synthetic construct
  source 1..722: mol_type = other DNA
gcatcaaattaagcagaaggccatcctgacggatggcctttttgcgtttgcaccagtgatcagggttattcccttgggaaatctattgagatattctatcactcaaatagcaatataaggactctctgaattcatgaaatttggaaactttttacttacataccaacctccccaattttctcaaacagaggtaatggaacgtttggttaaattaggtcgcatctctgaggagtgtggttttgataccgtatggttactggagcatcatttcacggagtttggtctacttggtaacccttatgtcgctgctgcatatttacttggcgcgactaaaaaattgaatgtaggaaccgccgctattgttcttcccacagcccatccagtacgccaacttgaagatgtgaatttattggatcaaatgtcaaaaggacgatttcggtttggtatttgccgagggctttacaacaaggactttcgcgtattcggcgcggatatgaataacagtcgcgccttagcggaatgctggtacgggctgataaagaatggcatgacagagggatatatggaagctgataatgaacatatcaagttccataaggtaaaagtaaaccccgcggcgtatagcagaggtggcgcaccggtttatgtggtggctgaatcagctgcgacgactgagtgggcagctcaatttggcctaatcagttctggaccagcgagctgtg

SEQ ID NO: 17 - DNA, 688 bp, synthetic construct
  source 1..688: mol_type = other DNA
gtttgagacgggcgacagataggccataaattgggtgctgaagtctttgcgagagattgtcttgattttggttgggaagatttagcttctacaattaataacatgatgtatcttgatataccgtttattgcttttactgcaaataacgataattgggtcaagcaagatgaagttatcacattgttatcaaatattcgtagtaatcgatgcaagatatattctttgttaggaagttcgcatgacttgagtgaaaatttagtggtcctgcgcaatttttatcaatcggttacgaaagccgctatcgcgatggataatgatcatctggatattgatgttgatattactgaaccgtcatttgaacatttaactattgcgacagtcaatgaacgccgaatgagaattgagattgaaaatcaagcaatttctctgtcttaatgcgggcaggttatgtaggttgagagatcgttgacggctagctcagtcctaggtacagtgctagctactagtgaaagaggagaaatactagatgtcgaagggcgaagagcttttcacaggggtagtaccaattttagtagagttggatggtgatgtaaatggtcataagttttcagtacgtggggaaggtgaaggggacgcgacaaatggtaaattgactcttaaatttatctgcactaccggcaaattaccc

SEQ ID NO: 18 - DNA, 642 bp, synthetic construct
  source 1..642: mol_type = other DNA
gcactaccggcaaattacccgttccatggcctactctggtgacgacactgacctacggtgttcagtgtttcagtcgttaccctgaccatatgaaacgccatgatttcttcaagtcggctatgcccgaaggctacgttcaggaacgtacaatttctttcaaggatgacgggacgtacaagactcgtgctgaagtaaagtttgaaggggatacattggtaaatcgtattgagttgaagggtattgactttaaggaggatggaaacatcttaggccataagcttgaatacaacttcaactcccacaacgtttatatcacagctgacaaacagaaaaacggaattaaagctaactttaagatccgccacaatgttgaagacggaagcgttcaattagcggatcactaccagcagaacacacccattggggatggacctgtcttgttgccagataatcattatctgagtacacaatccgtgttatcaaaggacccgaatgaaaagcgtgaccacatggtgctgcttgagtttgtcacagcggcagggatcacgcacggcatggacgaactgtacaagtaaaacggcccggagggtggcgggcaggacgcccgccataaactgccaggcatcaaattaagcagaaggc

SEQ ID NO: 19 - DNA, 716 bp, synthetic construct
  source 1..716: mol_type = other DNA
catcaaattaagcagaaggccatcctgacggatggcctttttgcgtttgcaccagtgatcagggttattcccttgggaaatctattgagatattctatcactcaaatagcaatataaggactctctgaattcatgaaatttggaaactttttacttacataccaacctccccaattttctcaaacagaggtaatggaacgtttggttaaattaggtcgcatctctgaggagtgtggttttgataccgtatggttactggagcatcatttcacggagtttggtctacttggtaacccttatgtcgctgctgcatatttacttggcgcgactaaaaaattgaatgtaggaaccgccgctattgttcttcccacagcccatccagtacgccaacttgaagatgtgaatttattggatcaaatgtcaaaaggacgatttcggtttggtatttgccgagggctttacaacaaggactttcgcgtattcggcgcggatatgaataacagtcgcgccttagcggaatgctggtacgggctgataaagaatggcatgacagagggatatatggaagctgataatgaacatatcaagttccataaggtaaaagtaaaccccgcggcgtatagcagaggtggcgcaccggtttatgtggtggctgaatcagctgcgacgactgagtgggcagctcaatttggcctaatcagttctggaccagcgag

SEQ ID NO: 20 - DNA, 681 bp, synthetic construct
  source 1..681: mol_type = other DNA
agacgggcgacagataggccataaattgggtgctgaagtctttgcgagagattgtcttgattttggttgggaagatttagcttctacaattaataacatgatgtatcttgatataccgtttattgcttttactgcaaataacgataattgggtcaagcaagatgaagttatcacattgttatcaaatattcgtagtaatcgatgcaagatatattctttgttaggaagttcgcatgacttgagtgaaaatttagtggtcctgcgcaatttttatcaatcggttacgaaagccgctatcgcgatggataatgatcatctggatattgatgttgatattactgaaccgtcatttgaacatttaactattgcgacagtcaatgaacgccgaatgagaattgagattgaaaatcaagcaatttctctgtcttaatgcgggcaggttatgtaggttgagagatcgttgacggctagctcagtcctaggtacagtgctagctactagtgaaagaggagaaatactagatgtcgaagggcgaagagcttttcacaggggtagtaccaattttagtagagttggatggtgatgtaaatggtcataagttttcagtacgtggggaaggtgaaggggacgcgacaaatggtaaattgactcttaaatttatctgcactaccggcaaattac

SEQ ID NO: 21 - DNA, 638 bp, synthetic construct
  source 1..638: mol_type = other DNA
ctaccggcaaattacccgttccatggcctactctggtgacgacactgacctacggtgttcagtgtttcagtcgttaccctgaccatatgaaacgccatgatttcttcaagtcggctatgcccgaaggctacgttcaggaacgtacaatttctttcaaggatgacgggacgtacaagactcgtgctgaagtaaagtttgaaggggatacattggtaaatcgtattgagttgaagggtattgactttaaggaggatggaaacatcttaggccataagcttgaatacaacttcaactcccacaacgtttatatcacagctgacaaacagaaaaacggaattaaagctaactttaagatccgccacaatgttgaagacggaagcgttcaattagcggatcactaccagcagaacacacccattggggatggacctgtcttgttgccagataatcattatctgagtacacaatccgtgttatcaaaggacccgaatgaaaagcgtgaccacatggtgctgcttgagtttgtcacagcggcagggatcacgcacggcatggacgaactgtacaagtaaaacggcccggagggtggcgggcaggacgcccgccataaactgccaggcatcaaattaagcagaagg

SEQ ID NO: 22 - DNA, 707 bp, synthetic construct
  source 1..707: mol_type = other DNA
aaattaagcagaaggccatcctgacggatggcctttttgcgtttgcaccagtgatcagggttattcccttgggaaatctattgagatattctatcactcaaatagcaatataaggactctctgaattcatgaaatttggaaactttttacttacataccaacctccccaattttctcaaacagaggtaatggaacgtttggttaaattaggtcgcatctctgaggagtgtggttttgataccgtatggttactggagcatcatttcacggagtttggtctacttggtaacccttatgtcgctgctgcatatttacttggcgcgactaaaaaattgaatgtaggaaccgccgctattgttcttcccacagcccatccagtacgccaacttgaagatgtgaatttattggatcaaatgtcaaaaggacgatttcggtttggtatttgccgagggctttacaacaaggactttcgcgtattcggcgcggatatgaataacagtcgcgccttagcggaatgctggtacgggctgataaagaatggcatgacagagggatatatggaagctgataatgaacatatcaagttccataaggtaaaagtaaaccccgcggcgtatagcagaggtggcgcaccggtttatgtggtggctgaatcagctgcgacgactgagtgggcagctcaatttggcctaatcagttctggacca

(19) United States
(12) Patent Application Publication
(10) Pub. No.: US 2023/0257790 A1
(43) Pub. Date: Aug. 17, 2023

(54) RECOMBINATION-BASED DNA ASSEMBLY METHODS AND COMPOSITIONS

(71) Applicant: INTEGRATED DNA TECHNOLOGIES, INC., Coralville, IA (US)

(72) Inventors: Timothy Tapscott, Coralville, IA (US); Adam Van Grootheest, Marion, IA (US)

(21) Appl. No.: 18/161,654

(22) Filed: Jan. 30, 2023

Related U.S. Application Data
(60) Provisional application No. 63/304,792, filed on Jan. 31, 2022.

(51) Int. Cl. C12P 19/34; C12Q 1/686

(52) U.S. Cl. CPC C12P 19/34; C12Q 1/686

(57) ABSTRACT

Described herein are compositions and methods for recombination-based assembly of long dsDNA molecules. One embodiment described herein is a method for creating large recombinant plasmids in a competent host cell using a plurality of double stranded DNA fragments containing overlapping fragments using a recombinase and exonuclease.

### CROSS-REFERENCE TO RELATED APPLICATIONS

This application claims priority to U.S. Provisional Patent Application No. 63/304,792 filed on Jan. 31, 2022, which is incorporated by reference herein in its entirety.

### REFERENCE TO SEQUENCE LISTING

This application was filed with a Sequence Listing XML in ST.26 XML format accordance with 37 C.F.R. § 1.831. The Sequence Listing XML file submitted in the USPTO Patent Center, 10 “013670-0012-WO01_sequence_listing_xml_18-JAN-2023.xml,” was created on Jan. 18, 2023, contains 22 sequences, has a file size of 45.6 Kbytes, and is incorporated by reference in its entirety into the specification.

### TECHNICAL FIELD

Described herein are compositions and methods for recombination-based assembly of long dsDNA molecules. One embodiment described herein is a method for creating large recombinant plasmids in a competent host cell using a plurality of double stranded DNA fragments containing overlapping fragments using a recombinase and exonuclease.

### BACKGROUND

Nucleic acid recombination lies at the core of molecular biology and biotechnology. The efficiency whereby recombinant nucleic acid technology is achieved can dictate the outcome of certain biotechnology implementations. The ability to perform DNA assembly or the ability to physically link multiple double stranded DNA (dsDNA) fragments together to generate longer dsDNA fragments is a key technology in synthetic biology.

What are needed are methods and compositions to overcome the existing challenges of current nucleic acid recombination technologies and reduce the complexity of workflows, increase DNA assembly efficiency and fidelity, while reducing overall assembly cost.

### SUMMARY

One embodiment described herein is a method for the assembly of a plurality of double stranded DNA (dsDNA) fragments into a covalently bound circular dsDNA molecule, the method comprising: (a) combining a plurality of distinct dsDNA fragments with a reaction mixture comprising an exonuclease and a recombinase to form a DNA reaction mixture; wherein each individual dsDNA fragment comprises one or more terminal single-stranded nucleotides that are complementary to terminal single-stranded nucleotides of an independent dsDNA fragment; (b) subjecting the DNA reaction mixture to a hybridization incubation to form a hybridized DNA reaction mixture; (c) subjecting the hybridized DNA reaction mixture to a deactivation incubation to form a deactivated DNA reaction mixture; (d) transforming the deactivated DNA reaction mixture into a competent host cell; and (e) incubating the transformed competent host cell under conditions sufficient to assemble and replicate one or more covalently bound circular dsDNA molecules comprising the plurality of distinct dsDNA fragments. In one aspect, the recombinase is selected from Uvsx from a bacteriophage, Rad51 or Dmc1 from a eukaryote, RadA from archaea, or RecA from E. coli. In another aspect, the recombinase is RecA from E. coli. In another aspect, the reaction mixture further comprises ATP. In another aspect, the exonuclease is T5 Exonuclease. In another aspect, the reaction mixture further comprises a DNA polymerase and a ligase. In another aspect, the competent host cell is an E. coli cell. In another aspect, the one or more terminal single-stranded nucleotides that are complementary overlap with terminal single-stranded nucleotides of the independent dsDNA fragment by about 10 nucleotides to about 120 nucleotides. In another aspect, the one or more terminal single-stranded nucleotides that are complementary overlap with terminal single-stranded nucleotides of the independent dsDNA fragment by about 20 nucleotides to about 60 nucleotides. In another aspect, the one or more terminal single-stranded nucleotides that are complementary overlap with terminal single-stranded nucleotides of the independent dsDNA fragment by about 20 nucleotides to about 35 nucleotides. In another aspect, the one or more terminal single-stranded nucleotides that are complementary overlap with terminal single-stranded nucleotides of the independent dsDNA fragment by about 25 nucleotides to about 30 nucleotides. In another aspect, the hybridization incubation comprises a hybridization temperature of about 25° C. to about 50° C. for about 5 minutes to about 120 minutes. In another aspect, the hybridization incubation comprises a hybridization temperature of about 35° C. to about 45° C. for about 10 minutes to about 20 minutes. In another aspect, the hybridization incubation comprises a hybridization temperature of about 42° C. for about 20 minutes. In another aspect, the deactivation incubation comprises a deactivation temperature of about 60° C. to about 70° C. for about 5 minutes to about 120 minutes. In another aspect, the deactivation incubation comprises a deactivation temperature of about 65° C. for about 20 minutes. In another aspect, the deactivation incubation comprises a deactivation temperature of less than about 5° C. for about 20 minutes. In another aspect, the reaction mixture further comprises one or more crowding agents, one or more chaperone agents, or a combination thereof. In another aspect, the one or more crowding agents comprises polyethylene glycol (PEG). In another aspect, the one or more chaperone agents comprises a diol or a polyol selected from substituted straight or branched alkylene glycols, pentaerythritol, sorbitol, diethylene glycol, dipropylene glycol, neopentyl glycol, propylene glycol and ethylene glycol ethers, 1,2-ethylene glycol, 1,2-PrD, 1,3-PrD, 1,4-butanediol, 1,5-pentanediol, 1,6-hexanediol, 2-methyl-1,3-propanediol, 2,2′-dimethylpropylene glycol, 1,3-butylethylpropanediol, methyl propanediol, methyl pentanediols, propylene glycol methyl ether, propylene glycol ethyl ether, propylene glycol butyl ether, diethylene glycol phenyl ether, propylene glycol phenol ether, propylene glycol methyl ether, tri-propylene glycol methyl ether, propylene glycol isobutyl ether, ethylene glycol methyl ether, or combinations thereof. In another aspect, the method further comprises isolating the covalently bound circular dsDNA molecules comprising the plurality of distinct dsDNA fragments from one or more competent host cells. In another aspect, the method further comprises sequencing the covalently bound circular DNA molecule comprising the plurality of distinct dsDNA fragments following isolation from the competent host cell.

Another embodiment described herein is a system for the assembly of a plurality of double stranded DNA (dsDNA) fragments into a covalently bound circular DNA molecule, the system comprising: (a) a plurality of distinct dsDNA fragments, wherein each individual dsDNA fragment comprises one or more terminal single-stranded nucleotides that are complementary to terminal single-stranded nucleotides of an independent dsDNA fragment from the plurality of distinct dsDNA fragments; (b) a reaction mixture comprising an exonuclease and a recombinase; and (c) a competent host cell. In another aspect, the recombinase is selected from Uvsx from a bacteriophage, Rad51 or Dmc1 from a eukaryote, RadA from archaea, or RecA from E. coli. In another aspect, the recombinase is RecA from E. coli. In another aspect, the reaction mixture further comprises ATP. In another aspect, the exonuclease is T5 Exonuclease. In another aspect, the reaction mixture further comprises a DNA polymerase and a ligase. In another aspect, the competent host cell is an E. coli cell. In another aspect, the reaction mixture further comprises one or more crowding agents, one or more chaperone agents, or a combination thereof.

Another embodiment described herein is a kit for the assembly of a plurality of double stranded DNA (dsDNA) fragments into a covalently bound circular DNA molecule, the kit comprising: (a) an exonuclease; (b) a recombinase; (c) one or more buffers, crowding agents, or chaperone agents, or ATP; (d) optionally, a competent host cell; and (e) optionally, instructions or directions for use.

Another embodiment described herein is the use of an exonuclease and a recombinase for the assembly of a plurality of double stranded DNA (dsDNA) fragments into a covalently bound circular DNA molecule, comprising: (a) combining a plurality of distinct dsDNA fragments with a reaction mixture comprising an exonuclease and a recombinase to form a DNA reaction mixture, wherein each individual dsDNA fragment comprises one or more terminal single-stranded nucleotides that are complementary to terminal single-stranded nucleotides of an independent dsDNA fragment from the plurality of distinct dsDNA fragments; (b) subjecting the DNA reaction mixture to a hybridization incubation to form a hybridized DNA reaction mixture; (c) subjecting the hybridized DNA reaction mixture to a deactivation incubation to form a deactivated DNA reaction mixture; (d) transforming the deactivated DNA reaction mixture into a competent host cell; and (e) incubating the transformed competent host cell under conditions sufficient to assemble a covalently bound circular DNA molecule comprising the plurality of distinct dsDNA fragments.

### DESCRIPTION OF THE DRAWINGS

FIG. 1A-1B show workflows for the assembly of dsDNA oligonucleotides. FIG. 1A shows the workflow for the assembly using exonuclease and recombinase. FIG. 1B shows the workflow for assembly using exonuclease, recombinase, ligase, and polymerase.

FIG. 2A-2B show illustrative schematics of the assembly of dsDNA oligonucleotides. FIG. 2A shows the assembly using exonuclease and recombinase (as shown in FIG. 1A). FIG. 2B shows the assembly using exonuclease, recombinase, ligase, and polymerase (as shown in FIG. 1B).

FIG. 3 shows total colony count with indicated reaction mix assembly compositions incubated with dsDNA fragments followed by transformation into the appropriate host cell. Error bars represent standard deviation.

FIG. 4 shows fluorescent colony count with indicated reaction mix assembly compositions incubated with dsDNA fragments followed by transformation into the appropriate host cell. Error bars represent standard deviation.

FIG. 5 shows percent of fluorescent colonies out of total colony count with indicated reaction mix assembly compositions incubated with dsDNA fragments followed by transformation into the appropriate host cell. Error bars represent standard deviation.

FIG. 6 shows fluorescent colony count with indicated reaction mix assembly compositions incubated with dsDNA fragments with and without heat denaturation prior to transformation into the appropriate host cell. Error bars represent standard deviation.

FIG. 7 shows fluorescent colony count using a recombinase and an exonuclease reaction mix incubated with dsDNA at indicated temperatures followed by transformation into the appropriate host cell. Error bars represent standard deviation.

FIG. 8 shows fluorescent colony count using a recombinase and an exonuclease reaction mix incubated with dsDNA with indicated length of sequence homology followed by transformation into the appropriate host cell. Error bars represent standard deviation.

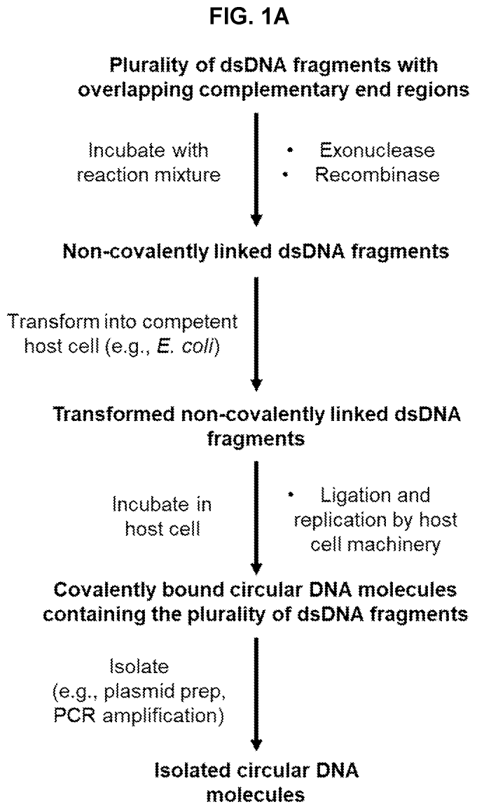

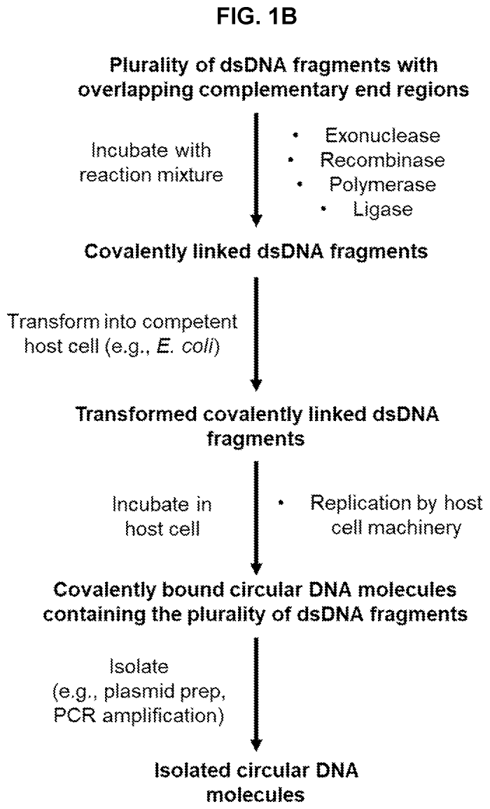

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “−j” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, the phrase “room temperature,” “RT,” or “ambient temperature” indicates a temperature of about 20-27° C.; about 25° C.±10%; or −25° C., at standard atmospheric pressure.

As used herein, the term “additive” refers to one or more components added to the buffers described herein to enhance hybridization and improve overall specificity.

As used herein, the term “nucleic acid” may refer to DNA, RNA, dsDNA, dsRNA, ssDNA, ssRNA, or hybrids of DNA/RNA complexes or sequences obtained from any source, containing target and non-target sequences. For example, a nucleic acid sample can be obtained from artificial sources or by chemical synthesis, or by enzymatic synthesis, or from viruses, prokaryotic cells including microbes, or eukaryotic cells. Biological samples may be vertebrate, including human or excluding humans, invertebrates, plants, microbes, viruses, mycoplasma, fungi, or Archaea.

As used herein, the terms “base pair” or “bp” refer to the interaction of one or more nucleotides in a single stranded nucleic acid molecule with one or more nucleotides in a complementary single stranded nucleic acid molecule via hydrogen bonding to form a double stranded nucleic acid molecule (e.g., double stranded DNA). The base pairs may be Watson-Crick, Hoogsteen, or other noncanonical base paring interactions. Typically, the base pairs are Watson-Crick base pairs, e.g., A-T (or A-U) with two hydrogen bonds, and C-G, with three hydrogen bonds.

As used herein, “complementary” refers to the ability of one nucleic acid to form base pairs with another nucleic acid. Typically, one strand runs 5′→3′ and pairs with complementary nucleotides in a second strand in the 3′→5′ direction. For example, 5′-GAATC-3′ is complementary to 5′-GATTC-3′ (i.e., 3′-CTTAG-5′) and can form a double stranded nucleic acid where each nucleotide forms a Watson-Crick base pair with its respective complement on the other strand.

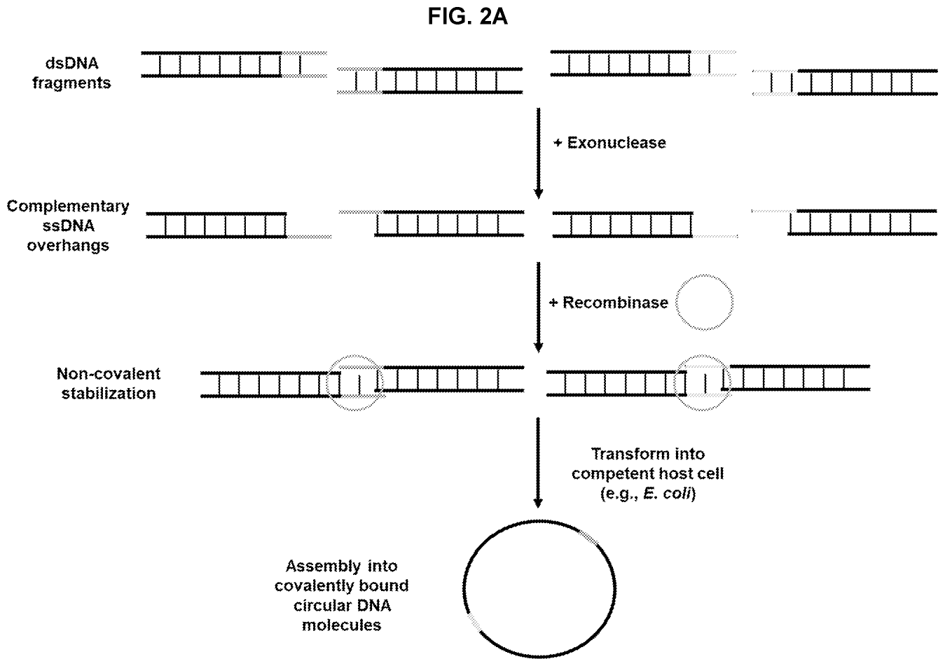

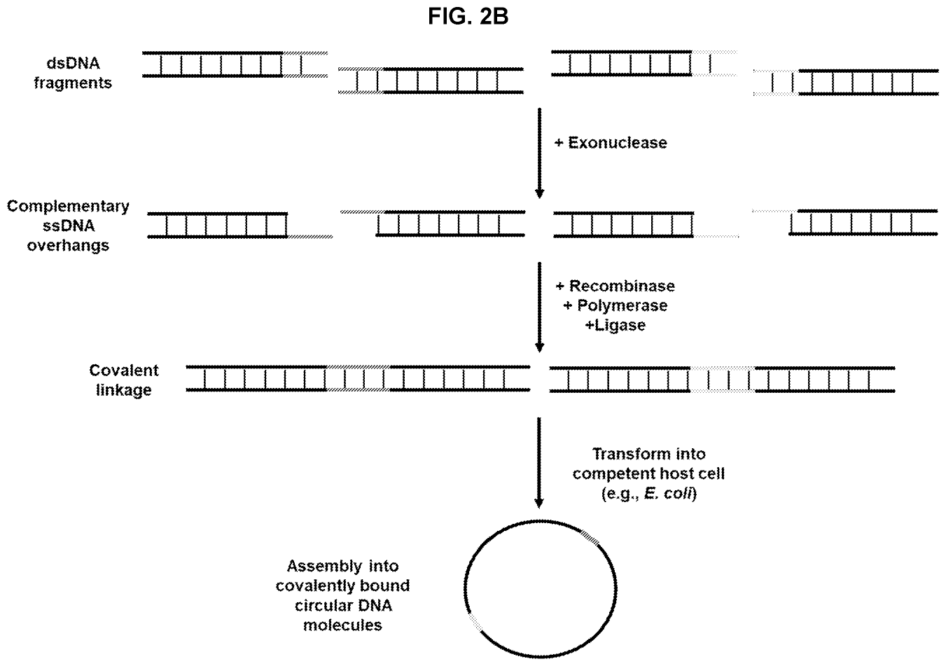

As used herein, the terms “overlap” or “overlapping” refer to double stranded nucleic acids that have non-base-paired nucleotides at one or both of the 5′- or 3′-termini (e.g., “overhangs”) that are capable of base-paring with complementary non-base paired nucleotides at one or both of the 5′- or 3′-termini of another double stranded sequence (e.g., “complementary overhangs”). See FIG. 2A-B which illustrate double stranded DNA molecules with overhanging single strands that overlap and are complementary to other distinct double strand molecules with overhanging single strands. In this manner, multiple distinct double stranded molecules with overhanging single strands on the 5′-terminus, 3′-terminus, or both, can be assembled to form long, non-covalently linked double stranded DNA molecules. See FIG. 2A.

As an illustrative example, a plurality of distinct dsDNA fragments could contain four dsDNA fragments, F1, F2, F3, and F4, each top strand oriented 5′-3′. To assemble a long dsDNA, fragment F1 would have a 3′-terminus that would overlap the 5′-terminus of F2. F2 would have a 5′-terminus that would overlap the 3′-terminus of F1 and a 3′-terminus that would overlap the 5′-terminus of F3. Likewise, F3 would have a 5′-terminus that would overlap the 3′-terminus of F2 and a 3′-terminus that would overlap the 5′-terminus of F4. F4 would have a 5′-terminus that would overlap the terminus of F3.

As used herein, the terms “long nucleic acid” or “large nucleic acids” refer to a nucleic acid that is greater than 100 nucleotides (or “base pairs” for double stranded nucleic acids). This includes single stranded nucleic acids, double stranded nucleic acids, plasmids, vectors, or other constructs known in the art. Long nucleic acids can be 100 to 20,000 nucleotides or greater, including all integers and subranges within the specified range. For example, long nucleic acids can include 100-1000, 100-5000, 500-2000, 1000-5000 nucleotides, or other subranges within 100-20,000 nucleotides.

As used herein, the terms “distinct” or “different” refer to double stranded DNA molecules or “fragments” that have different nucleotide sequences. For example, “a plurality of distinct dsDNA fragments” refers to multiple dsDNA molecules each having a particular sequence. In the plurality mixture, there may be multiple copies of each distinct dsDNA molecule, collectively forming a plurality of distinct dsDNA fragments. As used herein, the distinct dsDNA fragments may have sequences at one or both of the 5′- or 3′-termini that are capable of base-paring with complementary nucleotides at one or both of the 5′- or 3′-termini of another “independent” distinct dsDNA fragment (once the complementary regions are converted to single strands). As used herein, the term “independent” refers to a distinct dsDNA fragment that has a different dsDNA sequence as compared to another distinct dsDNA fragment.

Described herein are methods and compositions for improved DNA assembly of long double stranded nucleic acid molecules using a combination of in vitro alignment and in vivo methods. The methods are improved relative to the current state of the art.

Described herein are methods and compositions for a method of creating large recombinant plasmids in a competent host cell using a plurality of fragments of DNA containing sequences that overlap with adjacent fragments. In one aspect, the methods and compositions are useful for the assembly of two or more double-stranded DNA fragments using an exonuclease capable of creating single stranded overhangs from double stranded DNA and a recombinase to catalyze and stabilize complementary base pairing of the single-stranded (ssDNA) ends. This enables multi-fragmented dsDNA assemblies without the need for ligase or polymerase. While these assemblies are not covalently linked, the stabilization of the overlapping regions by the recombinase allows for transformation into E. coli followed by ligation and replication by the cellular machinery. In one aspect, the method comprises using a recombinase to stabilize the complementary regions to permit transformation into a cell and subsequent replication of the DNA to create a dsDNA replicate.

Additionally described herein are methods and compositions for assembly of double-stranded (dsDNA) fragments with a recombinase protein. In one aspect, the methods and compositions are useful for the assembly of two or more double-stranded DNA fragments using RecA to catalyze complementary base pairing of the single stranded DNA (ssDNA) ends in an ATP-dependent manner, enabling multi-fragmented dsDNA assemblies when performed in the presence of an exonuclease and optionally a polymerase and ligase.

One embodiment described herein is a reaction mixture of a plurality of distinct dsDNA fragments containing sequences that overlap with adjacent fragments by at least 10 base pairs, an exonuclease that creates single stranded overhangs of DNA, and a recombinase that facilitates the hybridization and stabilization of the newly created single strands into a competent cell host. Following the hybridization incubation at a temperature between 25° C. and 50° C. for 5 to 120 minutes and a subsequent deactivation incubation at 65° C. for 5 to 120 minutes, the mixture is transformed into a competent host cell. Following transformation and subsequent incubation a covalently bound circular molecule of DNA containing all the overlapping fragments is made. In one aspect, the reaction mixture optionally contains a crowding agent and/or a chaperone. In another aspect, the covalently bound circular DNA molecule containing the overlapping fragments is then isolated from the transformed cell. Exemplary methods include plasmid isolations or PCR amplification directly using the cell as the target nucleic acid. In another aspect, the sequences of the isolated fragments are verified by Sanger Sequencing or Next Generation Sequencing (NGS). Another aspect described herein, is a method for carrying out the reaction mixture, transforming cells, isolating the DNA, and sequencing the isolated DNA.

One embodiment described herein is a reaction mixture of a plurality of distinct dsDNA fragments containing sequences that overlap with adjacent fragments by 10 to 120 base pairs, an exonuclease that creates single stranded overhangs of DNA, and a recombinase that facilitates the hybridization of the newly created single strands, a polymerase that fills in gaps of single stranded DNA after the recombinase facilitated annealing, and a ligase that covalently bonds the fragments of DNA after fill in. Following the initial incubation at a constant temperature, the mixture is transformed into a competent host cell. Following transformation, a covalently bound circular molecule of DNA containing all the overlapping fragments is made. In another aspect, the reaction mixture further optionally contains a crowding agent, including but not limited to a polyethylene glycol (PEG). Another aspect described herein, are methods for performing the reaction mixture, transforming cells, isolating the DNA, and sequencing the isolated DNA.

Another embodiment described herein is a mixture of a plurality of distinct dsDNA fragments containing sequences that overlap with adjacent fragments by 10 to 120 base pairs, an exonuclease that creates single stranded overhangs of DNA, and a recombinase that facilitates the hybridization of the newly created single strands, a polymerase that fills in gaps of single stranded DNA after a recombinase facilitated annealing, and a ligase that covalently bonds the fragments of DNA after fill in. Following the initial incubation at a constant temperature, the mixture is transformed into a competent host cell. Following transformation, a covalently bound circular molecule of DNA containing all the overlapping fragments is made. In one aspect the mixture optionally contains a chaperone, including but not limited to, a diol or polyol. Examples of diols or polyols include optionally substituted straight or branched alkylene glycols, pentaerythritol, sorbitol, diethylene glycol, dipropylene glycol, neopentyl glycol, such as propylene glycol and ethylene glycol ethers, such as 1,2-ethylene glycol, 1,2-PrD, 1,3-PrD, 1,4-butanediol, 1,5-pentanediol, 1,6-hexanediol, 2-methyl-1,3-propanediol, 2,2′-dimethylpropylene glycol, 1,3-butylethylpropanediol, methyl propanediol, methyl pentanediols, propylene glycol methyl ether, propylene glycol ethyl ether, propylene glycol butyl ether, diethylene glycol phenyl ether, propylene glycol phenol ether, propylene glycol methyl ether, tri-propylene glycol methyl ether, propylene glycol isobutyl ether, ethylene glycol methyl ether, or mixtures thereof. Another aspect described herein, is a method for carrying out the reaction mixture, transforming cells, isolating the DNA, and sequencing the isolated DNA.

In one embodiment described herein the recombinase proteins comprise one or more of Uvsx from bacteriophages, Rad51 and Dmc1 From eukaryotes, RadA from archaea or RecA from E. coli. In another aspect, the recombinase proteins are ATP dependent. In another aspect, the recombinase is RecA or recA orthologs that contain DNA binding domains and promote DNA recombination.

In one embodiment described herein, the exonuclease is thermostable. In another aspect, the exonuclease is a 5′ to 3′ exonuclease or 3′ to 5′ exonuclease. In another aspect, the exonuclease is a 3′ to 5′ exonuclease. In yet another aspect, the exonuclease is T5 exonuclease.

In another embodiment described herein, the dsDNA nucleic acid fragments have overlapping ends. That is the 5′ and 3′ ends of adjoining fragments are complementary to each other. In one aspect the overlapping ends are 5 to 100 base pairs (bp), including all integers within and the endpoints of the specified range. In another aspect the overlapping ends are 5 bp, 10 bp, 15 bp, 20 bp, 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, or 100 bp. In another aspect the overlapping ends are between 10 bp and 60 bp. In another aspect the overlapping ends are 20 bp, 25 bp, 30 bp, 35 bp, 40 bp, 45 bp, or 50 bp.

In one embodiment, the dsDNA and reaction incubation time ranges from 10 minutes to 120 minutes, including all integers within and the endpoints of the specified range. In another aspect the deactivation incubation time ranges from 10 minutes to 120 minutes, including all integers within and the endpoints of the specified range. In another aspect, the hybridization conditions comprise an incubation temperature ranging from about 25° C. to about 50° C., including all integers within and the endpoints of the specified range. In another aspect, the deactivation conditions comprise an incubation temperature ranging from about 60° C. to about 70° C., including all integers within and the endpoints of the specified range. In another aspect, the deactivation conditions comprise an incubation temperature of about less than 5° C.

One embodiment described herein is a method for the assembly of a plurality of double stranded DNA (dsDNA) fragments into a covalently bound circular dsDNA molecule, the method comprising: (a) combining a plurality of distinct dsDNA fragments with a reaction mixture comprising an exonuclease and a recombinase to form a DNA reaction mixture; wherein each individual dsDNA fragment comprises one or more terminal single-stranded nucleotides that are complementary to terminal single-stranded nucleotides of an independent dsDNA fragment; (b) subjecting the DNA reaction mixture to a hybridization incubation to form a hybridized DNA reaction mixture; (c) subjecting the hybridized DNA reaction mixture to a deactivation incubation to form a deactivated DNA reaction mixture; (d) transforming the deactivated DNA reaction mixture into a competent host cell; and (e) incubating the transformed competent host cell under conditions sufficient to assemble and replicate one or more covalently bound circular dsDNA molecules comprising the plurality of distinct dsDNA fragments. In one aspect, the recombinase is selected from Uvsx from a bacteriophage, Rad51 or Dmc1 from a eukaryote, RadA from archaea, or RecA from E. coli. In another aspect, the recombinase is RecA from E. coli. In another aspect, the reaction mixture further comprises ATP. In another aspect, the exonuclease is T5 Exonuclease. In another aspect, the reaction mixture further comprises a DNA polymerase and a ligase. In another aspect, the competent host cell is an E. coli cell. In another aspect, the one or more terminal single-stranded nucleotides that are complementary overlap with terminal single-stranded nucleotides of the independent dsDNA fragment by about 10 nucleotides to about 120 nucleotides. In another aspect, the one or more terminal single-stranded nucleotides that are complementary overlap with terminal single-stranded nucleotides of the independent dsDNA fragment by about 20 nucleotides to about 60 nucleotides. In another aspect, the one or more terminal single-stranded nucleotides that are complementary overlap with terminal single-stranded nucleotides of the independent dsDNA fragment by about 20 nucleotides to about 35 nucleotides. In another aspect, the one or more terminal single-stranded nucleotides that are complementary overlap with terminal single-stranded nucleotides of the independent dsDNA fragment by about 25 nucleotides to about 30 nucleotides. In another aspect, the hybridization incubation comprises a hybridization temperature of about 25° C. to about 50° C. for about 5 minutes to about 120 minutes. In another aspect, the hybridization incubation comprises a hybridization temperature of about 35° C. to about 45° C. for about 10 minutes to about 20 minutes. In another aspect, the hybridization incubation comprises a hybridization temperature of about 42° C. for about 20 minutes. In another aspect, the deactivation incubation comprises a deactivation temperature of about 60° C. to about 70° C. for about 5 minutes to about 120 minutes. In another aspect, the deactivation incubation comprises a deactivation temperature of about 65° C. for about 20 minutes. In another aspect, the deactivation incubation comprises a deactivation temperature of less than about 5° C. for about 20 minutes. In another aspect, the reaction mixture further comprises one or more crowding agents, one or more chaperone agents, or a combination thereof. In another aspect, the one or more crowding agents comprises polyethylene glycol (PEG). In another aspect, the one or more chaperone agents comprises a diol or a polyol selected from substituted straight or branched alkylene glycols, pentaerythritol, sorbitol, diethylene glycol, dipropylene glycol, neopentyl glycol, propylene glycol and ethylene glycol ethers, 1,2-ethylene glycol, 1,2-PrD, 1,3-PrD, 1,4-butanediol, 1,5-pentanediol, 1,6-hexanediol, 2-methyl-1,3-propanediol, 2,2′-dimethylpropylene glycol, 1,3-butylethylpropanediol, methyl propanediol, methyl pentanediols, propylene glycol methyl ether, propylene glycol ethyl ether, propylene glycol butyl ether, diethylene glycol phenyl ether, propylene glycol phenol ether, propylene glycol methyl ether, tri-propylene glycol methyl ether, propylene glycol isobutyl ether, ethylene glycol methyl ether, or combinations thereof. In another aspect, the method further comprises isolating the covalently bound circular dsDNA molecules comprising the plurality of distinct dsDNA fragments from one or more competent host cells. In another aspect, the method further comprises sequencing the covalently bound circular DNA molecule comprising the plurality of distinct dsDNA fragments following isolation from the competent host cell.

Another embodiment described herein is a system for the assembly of a plurality of double stranded DNA (dsDNA) fragments into a covalently bound circular DNA molecule, the system comprising: (a) a plurality of distinct dsDNA fragments, wherein each individual dsDNA fragment comprises one or more terminal single-stranded nucleotides that are complementary to terminal single-stranded nucleotides of an independent dsDNA fragment from the plurality of distinct dsDNA fragments; (b) a reaction mixture comprising an exonuclease and a recombinase; and (c) a competent host cell. In another aspect, the recombinase is selected from Uvsx from a bacteriophage, Rad51 or Dmc1 from a eukaryote, RadA from archaea, or RecA from E. coli. In another aspect, the recombinase is RecA from E. coli. In another aspect, the reaction mixture further comprises ATP. In another aspect, the exonuclease is T5 Exonuclease. In another aspect, the reaction mixture further comprises a DNA polymerase and a ligase. In another aspect, the competent host cell is an E. coli cell. In another aspect, the reaction mixture further comprises one or more crowding agents, one or more chaperone agents, or a combination thereof.

Another embodiment described herein is a kit for the assembly of a plurality of double stranded DNA (dsDNA) fragments into a covalently bound circular DNA molecule, the kit comprising: (a) an exonuclease; (b) a recombinase; (c) one or more buffers, crowding agents, or chaperone agents, or ATP; (d) optionally, a competent host cell; and (e) optionally, instructions or directions for use.

Another embodiment described herein is the use of an exonuclease and a recombinase for the assembly of a plurality of double stranded DNA (dsDNA) fragments into a covalently bound circular DNA molecule, comprising: (a) combining a plurality of distinct dsDNA fragments with a reaction mixture comprising an exonuclease and a recombinase to form a DNA reaction mixture, wherein each individual dsDNA fragment comprises one or more terminal single-stranded nucleotides that are complementary to terminal single-stranded nucleotides of an independent dsDNA fragment from the plurality of distinct dsDNA fragments; (b) subjecting the DNA reaction mixture to a hybridization incubation to form a hybridized DNA reaction mixture; (c) subjecting the hybridized DNA reaction mixture to a deactivation incubation to form a deactivated DNA reaction mixture; (d) transforming the deactivated DNA reaction mixture into a competent host cell; and (e) incubating the transformed competent host cell under conditions sufficient to assemble a covalently bound circular DNA molecule comprising the plurality of distinct dsDNA fragments.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A method for the assembly of a plurality of double stranded DNA (dsDNA) fragments into a covalently bound circular dsDNA molecule, the method comprising:
   (a) combining a plurality of distinct dsDNA fragments with a reaction mixture comprising an exonuclease and a recombinase to form a DNA reaction mixture;
      wherein each individual dsDNA fragment comprises one or more terminal single-stranded nucleotides that are complementary to terminal single-stranded nucleotides of an independent dsDNA fragment;
   (b) subjecting the DNA reaction mixture to a hybridization incubation to form a hybridized DNA reaction mixture;
   (c) subjecting the hybridized DNA reaction mixture to a deactivation incubation to form a deactivated DNA reaction mixture;
   (d) transforming the deactivated DNA reaction mixture into a competent host cell; and
   (e) incubating the transformed competent host cell under conditions sufficient to assemble and replicate one or more covalently bound circular dsDNA molecules comprising the plurality of distinct dsDNA fragments.
Clause 2. The method of claim 1, wherein the recombinase is selected from Uvsx from a bacteriophage, Rad51 or Dmc1 from a eukaryote, RadA from archaea, or RecA from E. coli.
Clause 3. The method of claim 2, wherein the recombinase is RecA from E. coli.
Clause 4. The method of claim 1, wherein the reaction mixture further comprises ATP.
Clause 5. The method of claim 1, wherein the exonuclease is T5 Exonuclease.
Clause 6. The method of claim 1, wherein the reaction mixture further comprises a DNA polymerase and a ligase.
Clause 7. The method of claim 1, wherein the competent host cell is an E. coli cell.
Clause 8. The method of claim 1, wherein the one or more terminal single-stranded nucleotides that are complementary overlap with terminal single-stranded nucleotides of the independent dsDNA fragment by about 10 nucleotides to about 120 nucleotides.
Clause 9. The method of claim 8, wherein the one or more terminal single-stranded nucleotides that are complementary overlap with terminal single-stranded nucleotides of the independent dsDNA fragment by about 20 nucleotides to about 60 nucleotides.
Clause 10. The method of claim 9, wherein the one or more terminal single-stranded nucleotides that are complementary overlap with terminal single-stranded nucleotides of the independent dsDNA fragment by about 20 nucleotides to about 35 nucleotides.
Clause 11. The method of claim 10, wherein the one or more terminal single-stranded nucleotides that are complementary overlap with terminal single-stranded nucleotides of the independent dsDNA fragment by about 25 nucleotides to about 30 nucleotides.
Clause 12. The method of claim 1, wherein the hybridization incubation comprises a hybridization temperature of about 25° C. to about 50° C. for about 5 minutes to about 120 minutes.
Clause 13. The method of claim 12, wherein the hybridization incubation comprises a hybridization temperature of about 35° C. to about 45° C. for about 10 minutes to about 20 minutes.
Clause 14. The method of claim 13, wherein the hybridization incubation comprises a hybridization temperature of about 42° C. for about 20 minutes.
Clause 15. The method of claim 1, wherein the deactivation incubation comprises a deactivation temperature of about 60° C. to about 70° C. for about 5 minutes to about 120 minutes.
Clause 16. The method of claim 15, wherein the deactivation incubation comprises a deactivation temperature of about 65° C. for about 20 minutes.
Clause 17. The method of claim 1, wherein the deactivation incubation comprises a deactivation temperature of less than about 5° C. for about 20 minutes.
Clause 18. The method of claim 1, wherein the reaction mixture further comprises one or more crowding agents, one or more chaperone agents, or a combination thereof.
Clause 19. The method of claim 18, wherein the one or more crowding agents comprises polyethylene glycol (PEG).
Clause 20. The method of claim 18, wherein the one or more chaperone agents comprises a diol or a polyol selected from substituted straight or branched alkylene glycols, pentaerythritol, sorbitol, diethylene glycol, dipropylene glycol, neopentyl glycol, propylene glycol and ethylene glycol ethers, 1,2-ethylene glycol, 1,2-PrD, 1,3-PrD, 1,4-butanediol, 1,5-pentanediol, 1,6-hexanediol, 2-methyl-1,3-propanediol, 2,2′-dimethylpropylene glycol, 1,3-butylethylpropanediol, methyl propanediol, methyl pentanediols, propylene glycol methyl ether, propylene glycol ethyl ether, propylene glycol butyl ether, diethylene glycol phenyl ether, propylene glycol phenol ether, propylene glycol methyl ether, tri-propylene glycol methyl ether, propylene glycol isobutyl ether, ethylene glycol methyl ether, or combinations thereof.
Clause 21. The method of claim 1, further comprising isolating the covalently bound circular dsDNA molecules comprising the plurality of distinct dsDNA fragments from one or more competent host cells.
Clause 22. The method of claim 21, further comprising sequencing the covalently bound circular DNA molecule comprising the plurality of distinct dsDNA fragments following isolation from the competent host cell.
Clause 23. A system for the assembly of a plurality of double stranded DNA (dsDNA) fragments into a covalently bound circular DNA molecule, the system comprising:
   (a) a plurality of distinct dsDNA fragments, wherein each individual dsDNA fragment comprises one or more terminal single-stranded nucleotides that are complementary to terminal single-stranded nucleotides of an independent dsDNA fragment from the plurality of distinct dsDNA fragments;
   (b) a reaction mixture comprising an exonuclease and a recombinase; and
   (c) a competent host cell.
Clause 24. The system of claim 23, wherein the recombinase is selected from Uvsx from a bacteriophage, Rad51 or Dmc1 from a eukaryote, RadA from archaea, or RecA from E. coli.
Clause 25. The system of claim 24, wherein the recombinase is RecA from E. coli.
Clause 26. The system of claim 23, wherein the reaction mixture further comprises ATP.
Clause 27. The system of claim 23, wherein the exonuclease is T5 Exonuclease.
Clause 28. The system of claim 23, wherein the reaction mixture further comprises a DNA polymerase and a ligase.
Clause 29. The system of claim 23, wherein the competent host cell is an E. coli cell.
Clause 30. The system of claim 23, wherein the reaction mixture further comprises one or more crowding agents, one or more chaperone agents, or a combination thereof.
Clause 31. A kit for the assembly of a plurality of double stranded DNA (dsDNA) fragments into a covalently bound circular DNA molecule, the kit comprising:
   (a) an exonuclease;
   (b) a recombinase;
   (c) one or more buffers, crowding agents, or chaperone agents, or ATP;
   (d) optionally, a competent host cell; and
   (e) optionally, instructions or directions for use.
Clause 32. Use of an exonuclease and a recombinase for the assembly of a plurality of double stranded DNA (dsDNA) fragments into a covalently bound circular DNA molecule, comprising:
   (a) combining a plurality of distinct dsDNA fragments with a reaction mixture comprising an exonuclease and a recombinase to form a DNA reaction mixture, wherein each individual dsDNA fragment comprises one or more terminal single-stranded nucleotides that are complementary to terminal single-stranded nucleotides of an independent dsDNA fragment from the plurality of distinct dsDNA fragments;
   (b) subjecting the DNA reaction mixture to a hybridization incubation to form a hybridized DNA reaction mixture;
   (c) subjecting the hybridized DNA reaction mixture to a deactivation incubation to form a deactivated DNA reaction mixture;
   (d) transforming the deactivated DNA reaction mixture into a competent host cell; and
   (e) incubating the transformed competent host cell under conditions sufficient to assemble a covalently bound circular DNA molecule comprising the plurality of distinct dsDNA fragments.

### EXAMPLES

### Example 1

### Ligase, Polymerase, and Exonuclease Assembly

This example demonstrates dsDNA assembly using a reaction mix comprising an exonuclease, polymerase, and ligase.

A plurality of distinct dsDNA fragments with overlapping homologous ends were designed. See Table 1. 35 to 70 fmol of dsDNA fragments were added to a 20 μL reaction mix comprising 100 mM Tris·HCl, 10 mM MgCl2, 10 mM DTT, 1 M D-Sorbitol, 0.8 mM dNTPs, 0.004 U/μL T5 Exonuclease, 0.025 U/μL Polymerase, 0.2675 U/μL Ligase, 43 ng/μL. The plurality of distinct dsDNA fragments and reaction mix were incubated at 50° C. for 20 minutes, followed by incubation at 65° C. for 20 minutes. Following incubation, the reaction was transferred into competent E. coli DH5a cells and transformed via chemical transformation.

TABLE 1
Sequences Used
Vector with Target Gene (SEQ ID NO: 1)
TCGCGCGTTTCGGTGATGACGGTGAAAACCTCTGACACATGCAGCTCCCGGAGACGGTCACAGCTTGTCTGTAAGCGGATGCCG
GGAGCAGACAAGCCCGTCAGGGCGCGTCAGCGGGTGTTGGCGGGTGTCGGGGCTGGCTTAACTATGCGGCATCAGAGCAGATTG
TACTGAGAGTGCACCAAATGCGGTGTGAAATACCGCACAGATGCGTAAGGAGAAAATACCGCATCAGGCGCCATTCGCCATTCA
GGCTGCGCAACTGTTGGGAAGGGCGATCGGTGCGGGCCTCATCGCTATTACGCCAGCTGGCGAAAGGGGGATGTGCTGCAAGGC
GATTAAGTTGGGTAACGCCAGGGTTTTCCCAGTCACGACGTTGTAAAACGACGGCCAGTGCAACGCGATGACGATGGATAGCGA
TTCATCGATGAGCTGACCCGATCGCCGCCGCCGGAGGGTTGCGTTTGAGACGGGCGACAGATGTCGTACCGACTGGTAGATGAC
AGCAAACCTGCCCACATCGTCGCTGAACGAATTCCGTTCATACCTATTTTGTCACTTGAGCATCGACTTTGGGTTCCGTCGTTG
CCCGTTTTGCCAATGATCAACTTGCATGCTGTTCTGTCTGTCGCACTTGTTGCTTTATGATGAAGTGTCTTAGCACAATGTTAG
GATATACATGGCATTCAAATGAATTTGCGCGCGTGTGGCATCCCAGCTGGGTACTGTGTGCGTCGTCTGGCTGAGTTGGGAGTC
TTGTGACGGCGACCGGTCCTCTGCCGAAAATTTAACGCGGCGGCTTCTAAATCTCTACCGCTGCACTGCTGAGTTTGATTGTTC
GTCAATCCCGTCGTTGCGAACGCTTGACCGCGAAAAATTTGGGCATCGTTAGCGGATGAATCAGCTGAGACGTCGCACGTGGTC
AGCGCCGCTCGTTCATTCGCGTGCGCTGAACTATCGATAAAGAAATTCTTGGACGCGGCTGAGAAATCCAACTGTACGGTGGAC
ATTCCACCGCTGGTGCTTGTTAAATCATCCCTCTTGATATCGAACTTATTACATTGACAGACACGCGCAACGATATCCCCCCAG
TATGGTGCCCGCAATTGCGTCCTTAACGTTAAATGCGCCCGCGTTTCTCGGGTTTCCCATTTTCTTTTCGCCTTTTGTGGCTTA
AGTATGGTTTGCTTGATAACGCATCTCTGTCGAGTTCCCACCCTATCTTCGGTCACCGTGATGTTCGTCCGGTAAACATGAGCC
TTTACCCGGGCTGACTGTTCTCATAACACGGTTCCCGTGCCCATTAAATTATTGGGACCTTATTTGTACAAACATCGTCATTAG
GTCGCTTCGCTCCCCAGTTGATGCGTGTTTTGTGTCGCTCCGAAAGCGATATGCCCGCGAACCCAATTTGAACGTTAGGAAAGG
GGTGAACCGTCTTGTCGGTGGGGATCTTTCCGGTATTGGCCTTAATCTTGAGCTACGGACCCAAGATCGTGCGCACGACGTGAG
GTCCGTGAAACACGTGCTAATAAGGCTTCTGCGGCACAGGAAACGTCGCAGATACTCTTACGTAACGTACCGCAATTTCTTTTA
GCCTTTATTTACGTTTATTGGTCTCCTCTTTTATCAAATTTGCATACGTATTCATCTTACCAGAATGATTTGGAGTCAAAATGA
TGCATGGACATTGCCCCCATTCAGCACAAATTTGAGTAGGAGTCTCCTTGGTAGTCTTACACCCTATTTCCAATTTATTGGACC
GCCCCCGTACCTCTCGTGTTTGATAATCCGCCTTATTGTAACTTACAGTTACAATTCCCTATAAGTTCCAAGCGATTATGAATC
GTAAGGCTCTTCTGGATAGCTGGCCTGTGCCCCCTGCCGTGGTATCTCAACTGACTAGTCCTTGGATTAAGGCTATCTTGGGGT
CGGAGTTCTACTCAGATACTACCCAGAACACCGCCATTCGCTCTTCGTATTGGAAAACGCAACACATTCGTTCTTGGCCATTAC
CTGCCGTAGTTATCGGTCGTTGGGCGGTTCAAAATTTAGGGTTGGCGATTGCGATGGTGAAGGTTGGAACAGATCTGGTTGTTG
GTCGCTGTGGGGACGATTCCACAAGTCGTAAGAAGAATCTGATTTGACTGAAACTTAATCAGATGCTTTGGTCCACGACTACCA
TCTGAGCGGTCGGGTAATGCCTGAGTACCGATATGGTAATCAAGTTTGACAATGCAGTCACGCTATTCCTTTATCCATTTTAAC
ATGGTGAGCGCTTAGAGACCAGTTACTCCGAGTGCAGCTCCCGCGTACATATCTACAGCCGTTCGGCGGAAATGTAATCTCGTC
ATTAAGGTCGCTTGCCCCTTATTTTAAGCCGTGTGCGTCGTATGAATCGTCTGTGATCAGAGGGCGTCGTCATGCGCGACGCGG
TTTTCATTTTTTTCACCTGCATTCACATTTGTTCGCCGTGTCGCACCGACCGTACCAGTACGTTACCTCGTGTCTATCTGGTGG
GTTACTACGTTGATGCTCTTATGTGCGGGCAGGTTATGTAGGTTGAGAGATCGTTGACGGCTAGCTCAGTCCTAGGTACAGTGC
TAGCTACTAGTGAAAGAGGAGAAATACTAGATGTCGAAGGGCGAAGAGCTTTTCACAGGGGTAGTACCAATTTTAGTAGAGTTG
GATGGTGATGTAAATGGTCATAAGTTTTCAGTACGTGGGGAAGGTGAAGGGGACGCGACAAATGGTAAATTGACTCTTAAATTT
ATCTGCACTACCGGCAAATTACCCGTTCCATGGCCTACTCTGGTGACGACACTGACCTACGGTGTTCAGTGTTTCAGTCGTTAC
CCTGACCATATGAAACGCCATGATTTCTTCAAGTCGGCTATGCCCGAAGGCTACGTTCAGGAACGTACAATTTCTTTCAAGGAT
GACGGGACGTACAAGACTCGTGCTGAAGTAAAGTTTGAAGGGGATACATTGGTAAATCGTATTGAGTTGAAGGGTATTGACTTT
AAGGAGGATGGAAACATCTTAGGCCATAAGCTTGAATACAACTTCAACTCCCACAACGTTTATATCACAGCTGACAAACAGAAA
AACGGAATTAAAGCTAACTTTAAGATCCGCCACAATGTTGAAGACGGAAGCGTTCAATTAGCGGATCACTACCAGCAGAACACA
CCCATTGGGGATGGACCTGTCTTGTTGCCAGATAATCATTATCTGAGTACACAATCCGTGTTATCAAAGGACCCGAATGAAAAG
CGTGACCACATGGTGCTGCTTGAGTTTGTCACAGCGGCAGGGATCACGCACGGCATGGACGAACTGTACAAGTAAAACGGCCCG
GAGGGTGGCGGGCAGGACGCCCGCCATAAACTGCCAGGCATCAAATTAAGCAGAAGGCCATCCTGACGGATGGCCTTTTTGCGT
TTGCACCAGTGATCAGGGTTATTCCCTTGGGAGATCCGAAAACGGGCGTATAGTCGAGACCATCAGTTCTGGACCAGCGAGCTG
TGCTGCGACTCGTGGCGTAATCATGGTCATAGCTGTTTCCTGTGTGAAATTGTTATCCGCTCACAATTCCACACAACATACGAG
CCGGAAGCATAAAGTGTAAAGCCTGGGGTGCCTAATGAGTGAGCTAACTCACATTAATTGCGTTGCGCTCACTGCCCGCTTTCC
AGTCGGGAAACCTGTCGTGCCAGCTGCATTAATGAATCGGCCAACGCGCGGGGAGAGGCGGTTTGCGTATTGGGCGCTCTTCCG
CTTCCTCGCTCACTGACTCGCTGCGCTCGGTCGTTCGGCTGCGGCGAGCGGTATCAGCTCACTCAAAGGCGGTAATACGGTTAT
CCACAGAATCAGGGGATAACGCAGGAAAGAACATGTGAGCAAAAGGCCAGCAAAAGGCCAGGAACCGTAAAAAGGCCGCGTTGC
TGGCGTTTTTCCATAGGCTCCGCCCCCCTGACGAGCATCACAAAAATCGACGCTCAAGTCAGAGGTGGCGAAACCCGACAGGAC
TATAAAGATACCAGGCGTTTCCCCCTGGAAGCTCCCTCGTGCGCTCTCCTGTTCCGACCCTGTCGCTTACCGGATACCTGTCCG
CCTTTCTCCCTTCGGGAAGCGTGGCGCTTTCTCATAGCTCACGCTGTAGGTATCTCAGTTCGGTGTAGGTCGTTCGCTCCAAGC
TGGGCTGTGTGCACGAACCCCCCGTTCAGCCCGACCGCTGCGCCTTATCCGGTAACTATCGTCTTGAGTCCAACCCGGTAAGAC
ACGACTTATCGCCACTGGCAGCAGCCACTGGTAACAGGATTAGCAGAGCGAGGTATGTAGGCGGTGCTACAGAGTTCTTGAAGT
GGTGGCCTAACTACGGCTACACTAGAAGAACAGTATTTGGTATCTGCGCTCTGCTGAAGCCAGTTACCTTCGGAAAAAGAGTTG
GTAGCTCTTGATCCGGCAAACAAACCACCGCTGGTAGCGGTGGTTTTTTTGTTTGCAAGCAGCAGATTACGCGCAGAAAAAAAG
GATCTCAAGAAGATCCTTTGATCTTTTCTACGGGGTCTGACGCTCAGTGGAACGAAAACTCACGTTAAGGGATTTTGGTCATGA
GATTATCAAAAAGGATCTTCACCTAGATCCTTTTAAATTAAAAATGAAGTTTTAAATCAATCTAAAGTATATATGAGTAAACTT
GGTCTGACAGTTACCAATGCTTAATCAGTGAGGCACCTATCTCAGCGATCTGTCTATTTCGTTCATCCATAGTTGCCTGACTCC
CCGTCGTGTAGATAACTACGATACGGGAGGGCTTACCATCTGGCCCCAGTGCTGCAATGATACCGCGAGACCCACGCTCACCGG
CTCCAGATTTATCAGCAATAAACCAGCCAGCCGGAAGGGCCGAGCGCAGAAGTGGTCCTGCAACTTTATCCGCCTCCATCCAGT
CTATTAATTGTTGCCGGGAAGCTAGAGTAAGTAGTTCGCCAGTTAATAGTTTGCGCAACGTTGTTGCCATTGCTACAGGCATCG
TGGTGTCACGCTCGTCGTTTGGTATGGCTTCATTCAGCTCCGGTTCCCAACGATCAAGGCGAGTTACATGATCCCCCATGTTGT
GCAAAAAAGCGGTTAGCTCCTTCGGTCCTCCGATCGTTGTCAGAAGTAAGTTGGCCGCAGTGTTATCACTCATGGTTATGGCAG
CACTGCATAATTCTCTTACTGTCATGCCATCCGTAAGATGCTTTTCTGTGACTGGTGAGTACTCAACCAAGTCATTCTGAGAAT
AGTGTATGCGGCGACCGAGTTGCTCTTGCCCGGCGTCAATACGGGATAATACCGCGCCACATAGCAGAACTTTAAAAGTGCTCA
TCATTGGAAAACGTTCTTCGGGGCGAAAACTCTCAAGGATCTTACCGCTGTTGAGATCCAGTTCGATGTAACCCACTCGTGCAC
CCAACTGATCTTCAGCATCTTTTACTTTCACCAGCGTTTCTGGGTGAGCAAAAACAGGAAGGCAAAATGCCGCAAAAAAGGGAA
TAAGGGCGACACGGAAATGTTGAATACTCATACTCTACCTTTTTCAATATTATTGAAGCATTTATCAGGGTTATTGTCTCATGA
GCGGATACATATTTGAATGTATTTAGAAAAATAAACAAATAGGGGTTCCGCGCACATTTCCCCGAAAAGTGCCACCTGACGTCT
AAGAAACCATTATTATCATGACATTAACCTATAAAAATAGGCGTATCACGAGGCCCTTTCGTC
GFP Frag1 (SEQ ID NO: 2)
GGAGGGTTGCGTTTGAGACGGGCGACAGATGTCGTACCGACTGGTAGATGACAGCAAACCTGCCCACATCGTCGCTGAACGAAT
TCCGTTCATACCTATTTTGTCACTTGAGCATCGACTTTGGGTTCCGTCGTTGCCCGTTTTGCCAATGATCAACTTGCATGCTGT
TCTGTCTGTCGCACTTGTTGCTTTATGATGAAGTGTCTTAGCACAATGTTAGGATATACATGGCATTCAAATGAATTTGCGCGC
GTGTGGCATCCCAGCTGGGTACTGTGTGCGTCGTCTGGCTGAGTTGGGAGTCTTGTGACGGCGACCGGTCCTCTGCCGAAAATT
TAACGCGGCGGCTTCTAAATCTCTACCGCTGCACTGCTGAGTTTGATTGTTCGTCAATCCCGTCGTTGCGAACGCTTGACCGCG
AAAAATTTGGGCATCGTTAGCGGATGAATCAGCTGAGACGTCGCACGTGGTCAGCGCCGCTCGTTCATTCGCGTGCGCTGAACT
ATCGATAAAGAAATTCTTGGACGCGGCTGAGAAATCCAACTGTACGGTGGACATTCCACCGCTGGTGCTTGTTAAATCATCCCT
CTTGATATCGAACTTATTACATTGACAGACACGCGCAACGATATCCCCCCAGTATGGTGCCCGCAATTGCGTCCTTAACGTTAA
ATGCGCCCGCGTTTCTCGGGTTTCCCATTTTCTTTTCGCCTTTTGTGGCTTAAGTATGGTTTGCTTGATAACGCATCTCTGTCG
AGTTCCCACCCTATCTTCGGTCACCGTGATGTTCGTCCGGTAAACATGAGCCT
GFP Frag2 (SEQ ID NO: 3)
CCGTGATGTTCGTCCGGTAAACATGAGCCTTTACCCGGGCTGACTGTTCTCATAACACGGTTCCCGTGCCCATTAAATTATTGG
GACCTTATTTGTACAAACATCGTCATTAGGTCGCTTCGCTCCCCAGTTGATGCGTGTTTTGTGTCGCTCCGAAAGCGATATGCC
CGCGAACCCAATTTGAACGTTAGGAAAGGGGTGAACCGTCTTGTCGGTGGGGATCTTTCCGGTATTGGCCTTAATCTTGAGCTA
CGGACCCAAGATCGTGCGCACGACGTGAGGTCCGTGAAACACGTGCTAATAAGGCTTCTGCGGCACAGGAAACGTCGCAGATAC
TCTTACGTAACGTACCGCAATTTCTTTTAGCCTTTATTTACGTTTATTGGTCTCCTCTTTTATCAAATTTGCATACGTATTCAT
CTTACCAGAATGATTTGGAGTCAAAATGATGCATGGACATTGCCCCCATTCAGCACAAATTTGAGTAGGAGTCTCCTTGGTAGT
CTTACACCCTATTTCCAATTTATTGGACCGCCCCCGTACCTCTCGTGTTTGATAATCCGCCTTATTGTAACTTACAGTTACAAT
TCCCTATAAGTTCCAAGCGATTATGAATCGTAAGGCTCTTCTGGATAGCTGGCCTGTGCCCCCTGCCGTGGTATCTCAACTGAC
TAGTCCTTGGATTAAGGCTATCTTGGGGTCGGAGTTCTACTCAGATACTACCCAGAACACCGCCATTCGCTCTTCGTATTGGAA
AACGCAACACATTCGTTCTTGGCCATTACCTGCCGTAGTTATCGGTCGTTGGGCGGTTCAAAATTTAGGGTTGGCGATTGCGAT
GGTGAAGGTTGGAACAGATCTGGT
GFP Frag3 (SEQ ID NO: 4)
TGCGATGGTGAAGGTTGGAACAGATCTGGTTGTTGGTCGCTGTGGGGACGATTCCACAAGTCGTAAGAAGAATCTGATTTGACT
GAAACTTAATCAGATGCTTTGGTCCACGACTACCATCTGAGCGGTCGGGTAATGCCTGAGTACCGATATGGTAATCAAGTTTGA
CAATGCAGTCACGCTATTCCTTTATCCATTTTAACATGGTGAGCGCTTAGAGACCAGTTACTCCGAGTGCAGCTCCCGCGTACA
TATCTACAGCCGTTCGGCGGAAATGTAATCTCGTCATTAAGGTCGCTTGCCCCTTATTTTAAGCCGTGTGCGTCGTATGAATCG
TCTGTGATCAGAGGGCGTCGTCATGCGCGACGCGGTTTTCATTTTTTTCACCTGCATTCACATTTGTTCGCCGTGTCGCACCGA
CCGTACCAGTACGTTACCTCGTGTCTATCTGGTGGGTTACTACGTTGATGCTCTTATGTGCGGGCAGGTTATGTAGGTTGAGAG
ATCGTTGACGGCTAGCTCAGTCCTAGGTACAGTGCTAGCTACTAGTGAAAGAGGAGAAATACTAGATGTCGAAGGGCGAAGAGC
TTTTCACAGGGGTAGTACCAATTTTAGTAGAGTTGGATGGTGATGTAAATGGTCATAAGTTTTCAGTACGTGGGGAAGGTGAAG
GGGACGCGACAAATGGTAAATTGACTCTTAAATTTATCTGCACTACCGGCAAATTACCCGTTCCATGGCCTACTCTGGTGACGA
CACTGACCTACGGTGTTCAGTGTTTCAGTCGT
GFP Frag4 (SEQ ID NO: 5)
TGCGATGGTGAAGGTTGGAACAGATCTGGTTGTTGGTCGCTGTGGGGACGATTCCACAAGTCGTAAGAAGAATCTGATTTGACT
GAAACTTAATCAGATGCTTTGGTCCACGACTACCATCTGAGCGGTCGGGTAATGCCTGAGTACCGATATGGTAATCAAGTTTGA
CAATGCAGTCACGCTATTCCTTTATCCATTTTAACATGGTGAGCGCTTAGAGACCAGTTACTCCGAGTGCAGCTCCCGCGTACA
TATCTACAGCCGTTCGGCGGAAATGTAATCTCGTCATTAAGGTCGCTTGCCCCTTATTTTAAGCCGTGTGCGTCGTATGAATCG
TCTGTGATCAGAGGGCGTCGTCATGCGCGACGCGGTTTTCATTTTTTTCACCTGCATTCACATTTGTTCGCCGTGTCGCACCGA
CCGTACCAGTACGTTACCTCGTGTCTATCTGGTGGGTTACTACGTTGATGCTCTTATGTGCGGGCAGGTTATGTAGGTTGAGAG
ATCGTTGACGGCTAGCTCAGTCCTAGGTACAGTGCTAGCTACTAGTGAAAGAGGAGAAATACTAGATGTCGAAGGGCGAAGAGC
TTTTCACAGGGGTAGTACCAATTTTAGTAGAGTTGGATGGTGATGTAAATGGTCATAAGTTTTCAGTACGTGGGGAAGGTGAAG
GGGACGCGACAAATGGTAAATTGACTCTTAAATTTATCTGCACTACCGGCAAATTACCCGTTCCATGGCCTACTCTGGTGACGA
CACTGACCTACGGTGTTCAGTGTTTCAGTCGT
pUCIDT-AMP (SEQ ID NO: 6)
ATCAGTTCTGGACCAGCGAGCTGTGCTGCGACTCGTGGCGTAATCATGGTCATAGCTGTTTCCTGTGTGAAATTGTTATCCGCT
CACAATTCCACACAACATACGAGCCGGAAGCATAAAGTGTAAAGCCTGGGGTGCCTAATGAGTGAGCTAACTCACATTAATTGC
GTTGCGCTCACTGCCCGCTTTCCAGTCGGGAAACCTGTCGTGCCAGCTGCATTAATGAATCGGCCAACGCGCGGGGAGAGGCGG
TTTGCGTATTGGGCGCTCTTCCGCTTCCTCGCTCACTGACTCGCTGCGCTCGGTCGTTCGGCTGCGGCGAGCGGTATCAGCTCA
CTCAAAGGCGGTAATACGGTTATCCACAGAATCAGGGGATAACGCAGGAAAGAACATGTGAGCAAAAGGCCAGCAAAAGGCCAG
GAACCGTAAAAAGGCCGCGTTGCTGGCGTTTTTCCATAGGCTCCGCCCCCCTGACGAGCATCACAAAAATCGACGCTCAAGTCA
GAGGTGGCGAAACCCGACAGGACTATAAAGATACCAGGCGTTTCCCCCTGGAAGCTCCCTCGTGCGCTCTCCTGTTCCGACCCT
GTCGCTTACCGGATACCTGTCCGCCTTTCTCCCTTCGGGAAGCGTGGCGCTTTCTCATAGCTCACGCTGTAGGTATCTCAGTTC
GGTGTAGGTCGTTCGCTCCAAGCTGGGCTGTGTGCACGAACCCCCCGTTCAGCCCGACCGCTGCGCCTTATCCGGTAACTATCG
TCTTGAGTCCAACCCGGTAAGACACGACTTATCGCCACTGGCAGCAGCCACTGGTAACAGGATTAGCAGAGCGAGGTATGTAGG
CGGTGCTACAGAGTTCTTGAAGTGGTGGCCTAACTACGGCTACACTAGAAGAACAGTATTTGGTATCTGCGCTCTGCTGAAGCC
AGTTACCTTCGGAAAAAGAGTTGGTAGCTCTTGATCCGGCAAACAAACCACCGCTGGTAGCGGTGGTTTTTTTGTTTGCAAGCA
GCAGATTACGCGCAGAAAAAAAGGATCTCAAGAAGATCCTTTGATCTTTTCTACGGGGTCTGACGCTCAGTGGAACGAAAACTC
ACGTTAAGGGATTTTGGTCATGAGATTATCAAAAAGGATCTTCACCTAGATCCTTTTAAATTAAAAATGAAGTTTTAAATCAAT
CTAAAGTATATATGAGTAAACTTGGTCTGACAGTTACCAATGCTTAATCAGTGAGGCACCTATCTCAGCGATCTGTCTATTTCG
TTCATCCATAGTTGCCTGACTCCCCGTCGTGTAGATAACTACGATACGGGAGGGCTTACCATCTGGCCCCAGTGCTGCAATGAT
ACCGCGAGACCCACGCTCACCGGCTCCAGATTTATCAGCAATAAACCAGCCAGCCGGAAGGGCCGAGCGCAGAAGTGGTCCTGC
AACTTTATCCGCCTCCATCCAGTCTATTAATTGTTGCCGGGAAGCTAGAGTAAGTAGTTCGCCAGTTAATAGTTTGCGCAACGT
TGTTGCCATTGCTACAGGCATCGTGGTGTCACGCTCGTCGTTTGGTATGGCTTCATTCAGCTCCGGTTCCCAACGATCAAGGCG
AGTTACATGATCCCCCATGTTGTGCAAAAAAGCGGTTAGCTCCTTCGGTCCTCCGATCGTTGTCAGAAGTAAGTTGGCCGCAGT
GTTATCACTCATGGTTATGGCAGCACTGCATAATTCTCTTACTGTCATGCCATCCGTAAGATGCTTTTCTGTGACTGGTGAGTA
CTCAACCAAGTCATTCTGAGAATAGTGTATGCGGCGACCGAGTTGCTCTTGCCCGGCGTCAATACGGGATAATACCGCGCCACA
TAGCAGAACTTTAAAAGTGCTCATCATTGGAAAACGTTCTTCGGGGCGAAAACTCTCAAGGATCTTACCGCTGTTGAGATCCAG
TTCGATGTAACCCACTCGTGCACCCAACTGATCTTCAGCATCTTTTACTTTCACCAGCGTTTCTGGGTGAGCAAAAACAGGAAG
GCAAAATGCCGCAAAAAAGGGAATAAGGGCGACACGGAAATGTTGAATACTCATACTCTACCTTTTTCAATATTATTGAAGCAT
TTATCAGGGTTATTGTCTCATGAGCGGATACATATTTGAATGTATTTAGAAAAATAAACAAATAGGGGTTCCGCGCACATTTCC
CCGAAAAGTGCCACCTGACGTCTAAGAAACCATTATTATCATGACATTAACCTATAAAAATAGGCGTATCACGAGGCCCTTTCG
TCTCGCGCGTTTCGGTGATGACGGTGAAAACCTCTGACACATGCAGCTCCCGGAGACGGTCACAGCTTGTCTGTAAGCGGATGC
CGGGAGCAGACAAGCCCGTCAGGGCGCGTCAGCGGGTGTTGGCGGGTGTCGGGGCTGGCTTAACTATGCGGCATCAGAGCAGAT
TGTACTGAGAGTGCACCAAATGCGGTGTGAAATACCGCACAGATGCGTAAGGAGAAAATACCGCATCAGGCGCCATTCGCCATT
CAGGCTGCGCAACTGTTGGGAAGGGCGATCGGTGCGGGCCTCATCGCTATTACGCCAGCTGGCGAAAGGGGGATGTGCTGCAAG
GCGATTAAGTTGGGTAACGCCAGGGTTTTCCCAGTCACGACGTTGTAAAACGACGGCCAGTGCAACGCGATGACGATGGATAGC
GATTCATCGATGAGCTGACCCGATCGCCGCCGCCGGAGGGTTGCGTTTGAGACGGGCGACAGAT

In this experiment, five linear double stranded DNA fragments were combined as described above. When correctly assembled, the fragments created a pUC-based plasmid that expressed green fluorescent protein (GFP). Evidence of correct assembly is shown by resistance to ampicillin and the fluorescent phenotype of the bacterial colonies when grown on LB agar plates (1% peptone, 0.5% yeast extract, 0.5% NaCl, 1.2% agar; 100 μg/mL ampicillin).

The transformation steps comprised transferring 2 μL of the incubated reaction mix to 15 μL of competent cells followed by transformation using heat shock at 42° C. for 30 seconds. Post transformation, 125 μL of SOC medium (2% tryptone, 0.5% yeast extract, 10 mM NaCl, 2.5 mM KCl, 10 mM MgCl2, 10 mM MgSO₄, and 20 mM glucose) was added to the heat shock cells followed by a 37° C. incubation for 1 hour. After the 37° C. incubation, the transformed cell and SOC media mixture was plated onto LB agar plates and incubated at 37° C. for 16 hours. Following the plate incubation, the total cells were counted as well as counting cells expressing fluorescent protein.

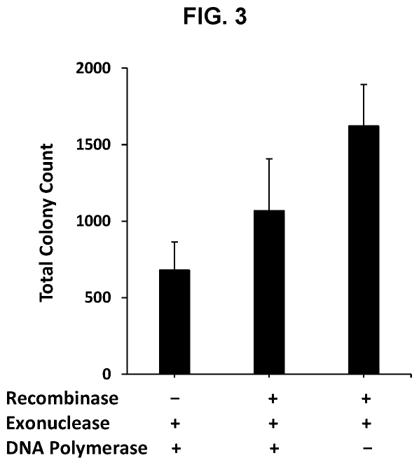

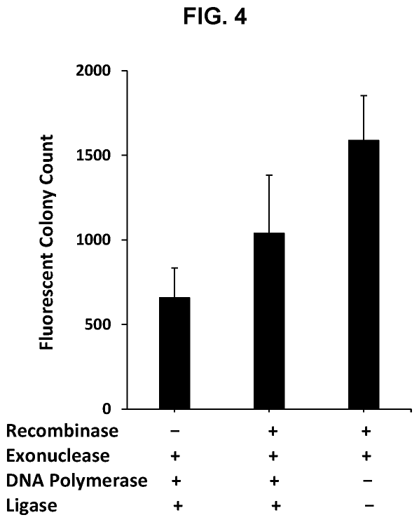

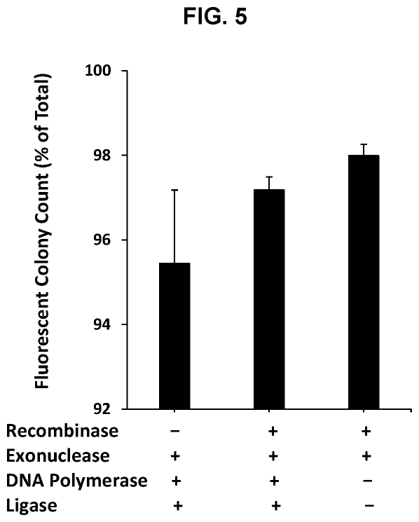

FIG. 3-5 show the total colony count, fluorescent colony count, and fluorescent colonies as a percentage, respectively. The first bar of each figure shows the counts for assembly mixes without recombinase (i.e., the reaction mix contains exonuclease, polymerase, and ligase enzymes).

### Example 2

### Recombinase, Ligase, Polymerase, and Exonuclease Assembly

This example demonstrates dsDNA assembly using a reaction mix comprising recombinase, exonuclease, polymerase, and ligase.

A plurality of distinct dsDNA fragments with overlapping homologous ends were designed. See Table 1 above. 35 to 70 fmol of dsDNA fragments were added to a 20 μL reaction mix comprising 100 mM Tris·HCl, 10 mM MgCl2, 10 mM DTT, 1 M D-Sorbitol, 0.8 mM dNTPs, 0.004 U/μL T5 Exonuclease, 0.025 U/μL Polymerase, 0.2675 U/μL Ligase, 43 ng/μL RecA, and 430 mM ATP. The plurality of distinct dsDNA fragments and reaction mix were incubated at 50° C. for 20 minutes, followed by incubation at 65° C. for 20 minutes. Following incubation, the reaction was transferred into competent E. coli DH5a cells via chemical transformation.

Five linear double stranded fragments of DNA were combined as described in Example 1; See Table 1 above. When correctly assembled, the fragments created a pUC-based plasmid that expressed green fluorescent protein (GFP). Evidence of correct assembly is shown by the resistance to ampicillin and the fluorescent phenotype of the bacterial colonies when grown on LB agar plates. The transformation steps comprised transferring 2 μL of the incubated reaction mix to 15 μL of competent cells followed by transformation using heat shock at 42° C. for 30 seconds. Post transformation, 125 μL of SOC media was added to the heat shock cells followed by a 37° C. incubation for 1 hour. After the 37° C. incubation, the transformed cell and SOC media mixture was plated onto LB agar plates and incubated at 37° C. for 16 hours. Following the plate incubation, the total cells were counted as well as counting cells expressing fluorescent protein.

FIG. 3-5 show the total colony count, fluorescent colony count, and fluorescent colonies as a percentage, respectively. The second bar of each figure shows the counts for assembly mixes containing recombinase. Additionally, the second bar of each figure shows improved assembly as compared to reaction mixes containing no recombinase, as shown in the first bar of each figure.

### Example 3

### Recombinase and Exonuclease Assembly

This example demonstrates dsDNA assembly techniques utilizing a reaction mix containing exonuclease and a recombinase. The reaction mix in this example does not contain ligase or polymerase.

A plurality of distinct dsDNA fragments with overlapping homologous ends were designed. See Table 1 above. 35-70 fmol of dsDNA fragments were added to a 20 μL reaction isothermal assembly reaction mix comprising 100 mM Tris·HCl, 10 mM MgCl2, 10 mM DTT, 1 M D-Sorbitol, 0.8 mM dNTPs, 0.004 U/μL T5 Exonuclease, 43 ng/μL RecA, and 430 mM ATP. The plurality of distinct dsDNA fragments and isothermal assembly reaction mix was incubated in a thermocycler at 50° C. for 20 minutes followed by incubation at 65° C. for 20 minutes. Following incubation, the reaction mix was transferred into DH5a E. coli cells via chemical competent transformation as described in Example 2.

FIG. 3-5 demonstrate the total colony count, fluorescent colony count, and fluorescent colonies as a percentage, respectively. The third bar of each figure shows the counts for assembly mixes with recombinase and exonuclease (i.e., not including polymerase or ligase).

FIG. 3-5 show that the recombinase and exonuclease assembly mix leads to an increase in total colony count, fluorescent colony count, and fluorescent colony percentage when compared to reaction mixes containing exonuclease, polymerase, and ligase (as shown in the first bar of each figure) and reaction mixes containing recombinase, exonuclease, polymerase, and ligase (as shown in the second bar of each figure).

### Example 4

### Recombinase and Exonuclease Assembly—Heat Denaturation

This example demonstrates that the hybridization between ssDNA ends and interaction with the recombinase are non-covalent and can be disrupted by heat denaturation.

Reactions were prepared as described in Example 3. The reactions were incubated in a thermocycler at 50° C. for 20 min, 65° C. for 20 min, with and without a 2 min 95° C. heat denaturation step. In the absence of the 2 min 95° C. heat denaturation step, the 65° C. incubation was extended by 2 min. Following incubation, the reaction mixtures were transferred into competent DH5a E. coli cells via chemical transformation as described in Example 2.

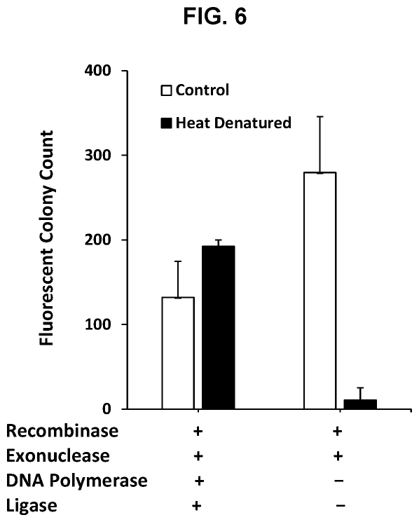

FIG. 6 shows that heat denaturation prior to transformation disrupts hybridization and interaction with the recombinase.

### Example 5

### Recombinase and Exonuclease Assembly—Temperature Testing

This example demonstrates the efficiency of the assembly recombinase and exonuclease assembly method under different hybridization incubation temperatures.

Reaction mix compositions were setup as previously described in Example 3 (reaction mixes contain recombinase and exonuclease with no polymerase or ligase) The plurality of distinct dsDNA fragments and reaction mix were incubated in a thermocycler at 25° C., 37° C., 42° C., or 50° C. for 20 min, followed by incubation at 65° C. for 20 min. Following incubation, the samples were transformed as described in Example 2.

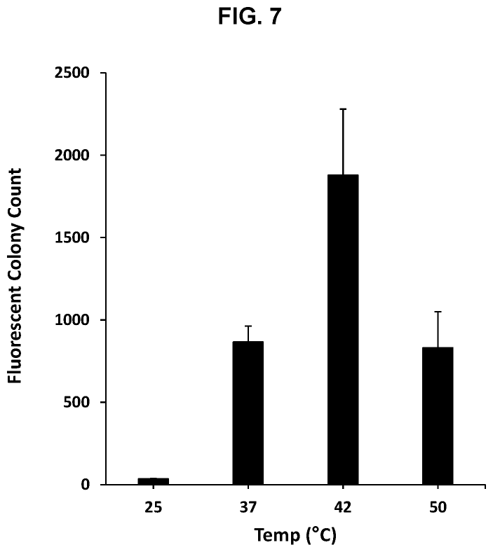

FIG. 7 demonstrates that the assembly reaction mix performs at a variety of incubation temperatures.

### Example 6

### Recombinase and Exonuclease Assembly Overhang Testing

This example demonstrates the effect of altered overhang length on the dsDNA fragments. The dsDNA fragments were designed such that the overhangs on the 3′- and 5′-ends of adjacent dsDNA fragments contained either 15 bp, 20 bp, 25 bp, 30 bp, or 35 bp of complementarity.

TABLE 2
Sequences Used
GFP3 (SEQ ID NO: 7)
TCGCGCGTTTCGGTGATGACGGTGAAAACCTCTGACACATGCAGCTCCCGGAGACGGTCACAGCTTGTCTGTAAGCGGATGCCG
GGAGCAGACAAGCCCGTCAGGGCGCGTCAGCGGGTGTTGGCGGGTGTCGGGGCTGGCTTAACTATGCGGCATCAGAGCAGATTG
TACTGAGAGTGCACCAAATGCGGTGTGAAATACCGCACAGATGCGTAAGGAGAAAATACCGCATCAGGCGCCATTCGCCATTCA
GGCTGCGCAACTGTTGGGAAGGGCGATCGGTGCGGGCCTCATCGCTATTACGCCAGCTGGCGAAAGGGGGATGTGCTGCAAGGC
GATTAAGTTGGGTAACGCCAGGGTTTTCCCAGTCACGACGTTGTAAAACGACGGCCAGTGCAACGCGATGACGATGGATAGCGA
TTCATCGATGAGCTGACCCGATCGCCGCCGCCGGAGGGTTGCGTTTGAGACGGGCGACAGATaggccataaattgggtgctgaa
gtctttgcgagagattgtcttgattttggttgggaagatttagcttctacaattaataacatgatgtatcttgatataccgttt
attgcttttactgcaaataacgataattgggtcaagcaagatgaagttatcacattgttatcaaatattcgtagtaatcgatgc
aagatatattctttgttaggaagttcgcatgacttgagtgaaaatttagtggtcctgcgcaatttttatcaatcggttacgaaa
gccgctatcgcgatggataatgatcatctggatattgatgttgatattactgaaccgtcatttgaacatttaactattgcgaca
gtcaatgaacgccgaatgagaattgagattgaaaatcaagcaatttctctgtcttaaTGCGGGCAGGTTATGTAGGTTGAGAGA
TCGttgacggctagctcagtcctaggtacagtgctagctactagtgaaagaggagaaatactagATGTCGAAGGGCGAAGAGCT
TTTCACAGGGGTAGTACCAATTTTAGTAGAGTTGGATGGTGATGTAAATGGTCATAAGTTTTCAGTACGTGGGGAAGGTGAAGG
GGACGCGACAAATGGTAAATTGACTCTTAAATTTATCTGCACTACCGGCAAATTACCCGTTCCATGGCCTACTCTGGTGACGAC
ACTGACCTACGGTGTTCAGTGTTTCAGTCGTTACCCTGACCATATGAAACGCCATGATTTCTTCAAGTCGGCTATGCCCGAAGG
CTACGTTCAGGAACGTACAATTTCTTTCAAGGATGACGGGACGTACAAGACTCGTGCTGAAGTAAAGTTTGAAGGGGATACATT
GGTAAATCGTATTGAGTTGAAGGGTATTGACTTTAAGGAGGATGGAAACATCTTAGGCCATAAGCTTGAATACAACTTCAACTC
CCACAACGTTTATATCACAGCTGACAAACAGAAAAACGGAATTAAAGCTAACTTTAAGATCCGCCACAATGTTGAAGACGGAAG
CGTTCAATTAGCGGATCACTACCAGCAGAACACACCCATTGGGGATGGACCTGTCTTGTTGCCAGATAATCATTATCTGAGTAC
ACAATCCGTGTTATCAAAGGACCCGAATGAAAAGCGTGACCACATGGTGCTGCTTGAGTTTGTCACAGCGGCAGGGATCACGCA
CGGCATGGACGAACTGTACAAGTAAaacggcccggaggggggggcaggacgcccgccataaactgccaggcatcaaattaag
cagaaggccatcctgacggatggcctttttgcgtttGCACCAGTGATCAGGGTTATTCCCTTGGGAaatctattgagatattct
atcactcaaatagcaatataaggactctctgaattcatgaaatttggaaactttttacttacataccaacctccccaattttct
caaacagaggtaatggaacgtttggttaaattaggtcgcatctctgaggagtgtggttttgataccgtatggttactggagcat
catttcacggagtttggtctacttggtaacccttatgtcgctgctgcatatttacttggcgcgactaaaaaattgaatgtagga
accgccgctattgttcttcccacagcccatccagtacgccaacttgaagatgtgaatttattggatcaaatgtcaaaaggacga
tttcggtttggtatttgccgagggctttacaacaaggactttcgcgtattcggcgcggatatgaataacagtcgcgccttagcg
gaatgctggtacgggctgataaagaatggcatgacagagggatatatggaagctgataatgaacatatcaagttccataaggta
aaagtaaaccccgcggcgtatagcagaggtggcgcaccggtttatgtggtggctgaatcagctgcgacgactgagtgggcagct
caatttggcctaATCAGTTCTGGACCAGCGAGCTGTGCTGCGACTCGTGGCGTAATCATGGTCATAGCTGTTTCCTGTGTGAAA
TTGTTATCCGCTCACAATTCCACACAACATACGAGCCGGAAGCATAAAGTGTAAAGCCTGGGGTGCCTAATGAGTGAGCTAACT
CACATTAATTGCGTTGCGCTCACTGCCCGCTTTCCAGTCGGGAAACCTGTCGTGCCAGCTGCATTAATGAATCGGCCAACGCGC
GGGGAGAGGCGGTTTGCGTATTGGGCGCTCTTCCGCTTCCTCGCTCACTGACTCGCTGCGCTCGGTCGTTCGGCTGCGGCGAGC
GGTATCAGCTCACTCAAAGGCGGTAATACGGTTATCCACAGAATCAGGGGATAACGCAGGAAAGAACATGTGAGCAAAAGGCCA
GCAAAAGGCCAGGAACCGTAAAAAGGCCGCGTTGCTGGCGTTTTTCCATAGGCTCCGCCCCCCTGACGAGCATCACAAAAATCG
ACGCTCAAGTCAGAGGTGGCGAAACCCGACAGGACTATAAAGATACCAGGCGTTTCCCCCTGGAAGCTCCCTCGTGCGCTCTCC
TGTTCCGACCCTGTCGCTTACCGGATACCTGTCCGCCTTTCTCCCTTCGGGAAGCGTGGCGCTTTCTCATAGCTCACGCTGTAG
GTATCTCAGTTCGGTGTAGGTCGTTCGCTCCAAGCTGGGCTGTGTGCACGAACCCCCCGTTCAGCCCGACCGCTGCGCCTTATC
CGGTAACTATCGTCTTGAGTCCAACCCGGTAAGACACGACTTATCGCCACTGGCAGCAGCCACTGGTAACAGGATTAGCAGAGC
GAGGTATGTAGGCGGTGCTACAGAGTTCTTGAAGTGGTGGCCTAACTACGGCTACACTAGAAGAACAGTATTTGGTATCTGCGC
TCTGCTGAAGCCAGTTACCTTCGGAAAAAGAGTTGGTAGCTCTTGATCCGGCAAACAAACCACCGCTGGTAGCGGTGGTTTTTT
TGTTTGCAAGCAGCAGATTACGCGCAGAAAAAAAGGATCTCAAGAAGATCCTTTGATCTTTTCTACGGGGTCTGACGCTCAGTG
GAACGAAAACTCACGTTAAGGGATTTTGGTCATGAGATTATCAAAAAGGATCTTCACCTAGATCCTTTTAAATTAAAAATGAAG
TTTTAAATCAATCTAAAGTATATATGAGTAAACTTGGTCTGACAGTTACCAATGCTTAATCAGTGAGGCACCTATCTCAGCGAT
CTGTCTATTTCGTTCATCCATAGTTGCCTGACTCCCCGTCGTGTAGATAACTACGATACGGGAGGGCTTACCATCTGGCCCCAG
TGCTGCAATGATACCGCGAGACCCACGCTCACCGGCTCCAGATTTATCAGCAATAAACCAGCCAGCCGGAAGGGCCGAGCGCAG
AAGTGGTCCTGCAACTTTATCCGCCTCCATCCAGTCTATTAATTGTTGCCGGGAAGCTAGAGTAAGTAGTTCGCCAGTTAATAG
TTTGCGCAACGTTGTTGCCATTGCTACAGGCATCGTGGTGTCACGCTCGTCGTTTGGTATGGCTTCATTCAGCTCCGGTTCCCA
ACGATCAAGGCGAGTTACATGATCCCCCATGTTGTGCAAAAAAGCGGTTAGCTCCTTCGGTCCTCCGATCGTTGTCAGAAGTAA
GTTGGCCGCAGTGTTATCACTCATGGTTATGGCAGCACTGCATAATTCTCTTACTGTCATGCCATCCGTAAGATGCTTTTCTGT
GACTGGTGAGTACTCAACCAAGTCATTCTGAGAATAGTGTATGCGGCGACCGAGTTGCTCTTGCCCGGCGTCAATACGGGATAA
TACCGCGCCACATAGCAGAACTTTAAAAGTGCTCATCATTGGAAAACGTTCTTCGGGGCGAAAACTCTCAAGGATCTTACCGCT
GTTGAGATCCAGTTCGATGTAACCCACTCGTGCACCCAACTGATCTTCAGCATCTTTTACTTTCACCAGCGTTTCTGGGTGAGC
AAAAACAGGAAGGCAAAATGCCGCAAAAAAGGGAATAAGGGCGACACGGAAATGTTGAATACTCATACTCTACCTTTTTCAATA
TTATTGAAGCATTTATCAGGGTTATTGTCTCATGAGCGGATACATATTTGAATGTATTTAGAAAAATAAACAAATAGGGGTTCC
GCGCACATTTCCCCGAAAAGTGCCACCTGACGTCTAAGAAACCATTATTATCATGACATTAACCTATAAAAATAGGCGTATCAC
GAGGCCCTTTCGTC
GFP3 35 bp overhangs
GFP3 35 Frag1 (SEQ ID NO: 8)
CCGCCGGAGGGTTGCGTTTGAGACGGGCGACAGATaggccataaattgggtgctgaagtctttgcgagagattgtcttgatttt
ggttgggaagatttagcttctacaattaataacatgatgtatcttgatataccgtttattgcttttactgcaaataacgataat
tgggtcaagcaagatgaagttatcacattgttatcaaatattcgtagtaatcgatgcaagatatattctttgttaggaagttcg
catgacttgagtgaaaatttagtggtcctgcgcaatttttatcaatcggttacgaaagccgctatcgcgatggataatgatcat
ctggatattgatgttgatattactgaaccgtcatttgaacatttaactattgcgacagtcaatgaacgccgaatgagaattgag
attgaaaatcaagcaatttctctgtcttaaTGCGGGCAGGTTATGTAGGTTGAGAGATCGttgacggctagctcagtcctaggt
acagtgctagctactagtgaaagaggagaaatactagATGTCGAAGGGCGAAGAGCTTTTCACAGGGGTAGTACCAATTTTAGT
AGAGTTGGATGGTGATGTAAATGGTCATAAGTTTTCAGTACGTGGGGAAGGTGAAGGGGACGCGACAAATGGTAAATTGACTCT
TAAATTTATCTGCACTACCGGCAAATTACCCGTTCCATGGC
GFP3 35 Frag2 (SEQ ID NO: 8)
TCTGCACTACCGGCAAATTACCCGTTCCATGGCCTACTCTGGTGACGACACTGACCTACGGTGTTCAGTGTTTCAGTCGTTACC
CTGACCATATGAAACGCCATGATTTCTTCAAGTCGGCTATGCCCGAAGGCTACGTTCAGGAACGTACAATTTCTTTCAAGGATG
ACGGGACGTACAAGACTCGTGCTGAAGTAAAGTTTGAAGGGGATACATTGGTAAATCGTATTGAGTTGAAGGGTATTGACTTTA
AGGAGGATGGAAACATCTTAGGCCATAAGCTTGAATACAACTTCAACTCCCACAACGTTTATATCACAGCTGACAAACAGAAAA
ACGGAATTAAAGCTAACTTTAAGATCCGCCACAATGTTGAAGACGGAAGCGTTCAATTAGCGGATCACTACCAGCAGAACACAC
CCATTGGGGATGGACCTGTCTTGTTGCCAGATAATCATTATCTGAGTACACAATCCGTGTTATCAAAGGACCCGAATGAAAAGC
GTGACCACATGGTGCTGCTTGAGTTTGTCACAGCGGCAGGGATCACGCACGGCATGGACGAACTGTACAAGTAAaacggcccgg
agggtggcgggcaggacgcccgccataaactgccaggcatcaaattaagcagaaggccatcctgacggat
GFP3 35 Frag3 (SEQ ID NO: 10)
gcatcaaattaagcagaaggccatcctgacggatggcctttttgcgtttGCACCAGTGATCAGGGTTATTCCCTTGGGAaatct
attgagatattctatcactcaaatagcaatataaggactctctgaattcatgaaatttggaaactttttacttacataccaacc
tccccaattttctcaaacagaggtaatggaacgtttggttaaattaggtcgcatctctgaggagtgtggttttgataccgtatg
gttactggagcatcatttcacggagtttggtctacttggtaacccttatgtcgctgctgcatatttacttggcgcgactaaaaa
attgaatgtaggaaccgccgctattgttcttcccacagcccatccagtacgccaacttgaagatgtgaatttattggatcaaat
gtcaaaaggacgatttcggtttggtatttgccgagggctttacaacaaggactttcgcgtattcggcgcggatatgaataacag
tcgcgccttagcggaatgctggtacgggctgataaagaatggcatgacagagggatatatggaagctgataatgaacatatcaa
gttccataaggtaaaagtaaaccccgcggcgtatagcagaggtggcgcaccggtttatgtggtggctgaatcagctgcgacgac
tgagtgggcagctcaatttggcctaATCAGTTCTGGACCAGCGAGCTGTGCTGCGACTCG
GFP3 30 bp overhangs
GFP3 30 Frag1 (SEQ ID NO: 11)
GGAGGGTTGCGTTTGAGACGGGCGACAGATaggccataaattgggtgctgaagtctttgcgagagattgtcttgattttggttg
ggaagatttagcttctacaattaataacatgatgtatcttgatataccgtttattgcttttactgcaaataacgataattgggt
caagcaagatgaagttatcacattgttatcaaatattcgtagtaatcgatgcaagatatattctttgttaggaagttcgcatga
cttgagtgaaaatttagtggtcctgcgcaatttttatcaatcggttacgaaagccgctatcgcgatggataatgatcatctgga
tattgatgttgatattactgaaccgtcatttgaacatttaactattgcgacagtcaatgaacgccgaatgagaattgagattga
aaatcaagcaatttctctgtcttaaTGCGGGCAGGTTATGTAGGTTGAGAGATCGttgacggctagctcagtcctaggtacagt
gctagctactagtgaaagaggagaaatactagATGTCGAAGGGCGAAGAGCTTTTCACAGGGGTAGTACCAATTTTAGTAGAGT
TGGATGGTGATGTAAATGGTCATAAGTTTTCAGTACGTGGGGAAGGTGAAGGGGACGCGACAAATGGTAAATTGACTCTTAAAT
TTATCTGCACTACCGGCAAATTACCCGTTCCAT
GFP3 30 Frag2 (SEQ ID NO: 12)
TCTGCACTACCGGCAAATTACCCGTTCCATGGCCTACTCTGGTGACGACACTGACCTACGGTGTTCAGTGTTTCAGTCGTTACC
CTGACCATATGAAACGCCATGATTTCTTCAAGTCGGCTATGCCCGAAGGCTACGTTCAGGAACGTACAATTTCTTTCAAGGATG
ACGGGACGTACAAGACTCGTGCTGAAGTAAAGTTTGAAGGGGATACATTGGTAAATCGTATTGAGTTGAAGGGTATTGACTTTA
AGGAGGATGGAAACATCTTAGGCCATAAGCTTGAATACAACTTCAACTCCCACAACGTTTATATCACAGCTGACAAACAGAAAA
ACGGAATTAAAGCTAACTTTAAGATCCGCCACAATGTTGAAGACGGAAGCGTTCAATTAGCGGATCACTACCAGCAGAACACAC
CCATTGGGGATGGACCTGTCTTGTTGCCAGATAATCATTATCTGAGTACACAATCCGTGTTATCAAAGGACCCGAATGAAAAGC
GTGACCACATGGTGCTGCTTGAGTTTGTCACAGCGGCAGGGATCACGCACGGCATGGACGAACTGTACAAGTAAaacggcccgg
agggtggcgggcaggacgcccgccataaactgccaggcatcaaattaagcagaaggccatcctgac
GFP3 30 Frag3 (SEQ ID NO: 13)
gcatcaaattaagcagaaggccatcctgacggatggcctttttgcgtttGCACCAGTGATCAGGGTTATTCCCTTGGGAaatct
attgagatattctatcactcaaatagcaatataaggactctctgaattcatgaaatttggaaactttttacttacataccaacc
tccccaattttctcaaacagaggtaatggaacgtttggttaaattaggtcgcatctctgaggagtgtggttttgataccgtatg
gttactggagcatcatttcacggagtttggtctacttggtaacccttatgtcgctgctgcatatttacttggcgcgactaaaaa
attgaatgtaggaaccgccgctattgttcttcccacagcccatccagtacgccaacttgaagatgtgaatttattggatcaaat
gtcaaaaggacgatttcggtttggtatttgccgagggctttacaacaaggactttcgcgtattcggcgcggatatgaataacag
togcgccttagcggaatgctggtacgggctgataaagaatggcatgacagagggatatatggaagctgataatgaacatatcaa
gttccataaggtaaaagtaaaccccgcggcgtatagcagaggtggcgcaccggtttatgtggtggctgaatcagctgcgacgac
tgagtgggcagctcaatttggcctaATCAGTTCTGGACCAGCGAGCTGTGCTGCG
GFP3 25 bp overhangs
GFP3 25 Frag1 (SEQ ID NO: 14)
GTTGCGTTTGAGACGGGCGACAGATaggccataaattgggtgctgaagtctttgcgagagattgtcttgattttggttgggaag
atttagcttctacaattaataacatgatgtatcttgatataccgtttattgcttttactgcaaataacgataattgggtcaagc
aagatgaagttatcacattgttatcaaatattcgtagtaatcgatgcaagatatattctttgttaggaagttcgcatgacttga
gtgaaaatttagtggtcctgcgcaatttttatcaatcggttacgaaagccgctatcgcgatggataatgatcatctggatattg
atgttgatattactgaaccgtcatttgaacatttaactattgcgacagtcaatgaacgccgaatgagaattgagattgaaaatc
aagcaatttctctgtcttaaTGCGGGCAGGTTATGTAGGTTGAGAGATCGttgacggctagctcagtcctaggtacagtgctag
ctactagtgaaagaggagaaatactagATGTCGAAGGGCGAAGAGCTTTTCACAGGGGTAGTACCAATTTTAGTAGAGTTGGAT
GGTGATGTAAATGGTCATAAGTTTTCAGTACGTGGGGAAGGTGAAGGGGACGCGACAAATGGTAAATTGACTCTTAAATTTATC
TGCACTACCGGCAAATTACCCGTTCC
GFP3 25 Frag2 (SEQ ID NO: 15)
GCACTACCGGCAAATTACCCGTTCCATGGCCTACTCTGGTGACGACACTGACCTACGGTGTTCAGTGTTTCAGTCGTTACCCTG
ACCATATGAAACGCCATGATTTCTTCAAGTCGGCTATGCCCGAAGGCTACGTTCAGGAACGTACAATTTCTTTCAAGGATGACG
GGACGTACAAGACTCGTGCTGAAGTAAAGTTTGAAGGGGATACATTGGTAAATCGTATTGAGTTGAAGGGTATTGACTTTAAGG
AGGATGGAAACATCTTAGGCCATAAGCTTGAATACAACTTCAACTCCCACAACGTTTATATCACAGCTGACAAACAGAAAAACG
GAATTAAAGCTAACTTTAAGATCCGCCACAATGTTGAAGACGGAAGCGTTCAATTAGCGGATCACTACCAGCAGAACACACCCA
TTGGGGATGGACCTGTCTTGTTGCCAGATAATCATTATCTGAGTACACAATCCGTGTTATCAAAGGACCCGAATGAAAAGCGTG
ACCACATGGTGCTGCTTGAGTTTGTCACAGCGGCAGGGATCACGCACGGCATGGACGAACTGTACAAGTAAaacggcccggagg
gtggcgggcaggacgcccgccataaactgccaggcatcaaattaagcagaaggccatc
GFP3 25 Frag3 (SEQ ID NO: 16)
gcatcaaattaagcagaaggccatcctgacggatggcctttttgcgtttGCACCAGTGATCAGGGTTATTCCCTTGGGAaatct
attgagatattctatcactcaaatagcaatataaggactctctgaattcatgaaatttggaaactttttacttacataccaacc
tccccaattttctcaaacagaggtaatggaacgtttggttaaattaggtcgcatctctgaggagtgtggttttgataccgtatg
gttactggagcatcatttcacggagtttggtctacttggtaacccttatgtcgctgctgcatatttacttggcgcgactaaaaa
attgaatgtaggaaccgccgctattgttcttcccacagcccatccagtacgccaacttgaagatgtgaatttattggatcaaat
gtcaaaaggacgatttcggtttggtatttgccgagggctttacaacaaggactttcgcgtattcggcgcggatatgaataacag
tegegccttagcggaatgctggtacgggctgataaagaatggcatgacagagggatatatggaagctgataatgaacatatcaa
gttccataaggtaaaagtaaaccccgcggcgtatagcagaggtggcgcaccggtttatgtggtggctgaatcagctgcgacgac
tgagtgggcagctcaatttggcctaATCAGTTCTGGACCAGCGAGCTGTG
GFP3 20 bp overhangs
GFP3 20 Frag1 (SEQ ID NO: 17)
GTTTGAGACGGGCGACAGATaggccataaattgggtgctgaagtctttgcgagagattgtcttgattttggttgggaagattta
gcttctacaattaataacatgatgtatcttgatataccgtttattgcttttactgcaaataacgataattgggtcaagcaagat
gaagttatcacattgttatcaaatattcgtagtaatcgatgcaagatatattctttgttaggaagttcgcatgacttgagtgaa
aatttagtggtcctgcgcaatttttatcaatcggttacgaaagccgctatcgcgatggataatgatcatctggatattgatgtt
gatattactgaaccgtcatttgaacatttaactattgcgacagtcaatgaacgccgaatgagaattgagattgaaaatcaagca
atttctctgtcttaaTGCGGGCAGGTTATGTAGGTTGAGAGATCGttgacggctagctcagtcctaggtacagtgctagctact
agtgaaagaggagaaatactagATGTCGAAGGGCGAAGAGCTTTTCACAGGGGTAGTACCAATTTTAGTAGAGTTGGATGGTGA
TGTAAATGGTCATAAGTTTTCAGTACGTGGGGAAGGTGAAGGGGACGCGACAAATGGTAAATTGACTCTTAAATTTATCTGCAC
TACCGGCAAATTACCC
GFP3 20 Frag2 (SEQ ID NO: 18)
GCACTACCGGCAAATTACCCGTTCCATGGCCTACTCTGGTGACGACACTGACCTACGGTGTTCAGTGTTTCAGTCGTTACCCTG
ACCATATGAAACGCCATGATTTCTTCAAGTCGGCTATGCCCGAAGGCTACGTTCAGGAACGTACAATTTCTTTCAAGGATGACG
GGACGTACAAGACTCGTGCTGAAGTAAAGTTTGAAGGGGATACATTGGTAAATCGTATTGAGTTGAAGGGTATTGACTTTAAGG
AGGATGGAAACATCTTAGGCCATAAGCTTGAATACAACTTCAACTCCCACAACGTTTATATCACAGCTGACAAACAGAAAAACG
GAATTAAAGCTAACTTTAAGATCCGCCACAATGTTGAAGACGGAAGCGTTCAATTAGCGGATCACTACCAGCAGAACACACCCA
TTGGGGATGGACCTGTCTTGTTGCCAGATAATCATTATCTGAGTACACAATCCGTGTTATCAAAGGACCCGAATGAAAAGCGTG
ACCACATGGTGCTGCTTGAGTTTGTCACAGCGGCAGGGATCACGCACGGCATGGACGAACTGTACAAGTAAaacggcccggagg
gtggcgggcaggacgcccgccataaactgccaggcatcaaattaagcagaaggc
GFP3 20 Frag3 (SEQ ID NO: 19)
catcaaattaagcagaaggccatcctgacggatggcctttttgcgtttGCACCAGTGATCAGGGTTATTCCCTTGGGAaatcta
ttgagatattctatcactcaaatagcaatataaggactctctgaattcatgaaatttggaaactttttacttacataccaacct
ccccaattttctcaaacagaggtaatggaacgtttggttaaattaggtcgcatctctgaggagtgtggttttgataccgtatgg
ttactggagcatcatttcacggagtttggtctacttggtaacccttatgtcgctgctgcatatttacttggcgcgactaaaaaa
ttgaatgtaggaaccgccgctattgttcttcccacagcccatccagtacgccaacttgaagatgtgaatttattggatcaaatg
tcaaaaggacgatttcggtttggtatttgccgagggctttacaacaaggactttcgcgtattcggcgcggatatgaataacagt
cgcgccttagcggaatgctggtacgggctgataaagaatggcatgacagagggatatatggaagctgataatgaacatatcaag
ttccataaggtaaaagtaaaccccgcggcgtatagcagaggtggcgcaccggtttatgtggtggctgaatcagctgcgacgact
gagtgggcagctcaatttggcctaATCAGTTCTGGACCAGCGAG
GFP3 15 bp overhangs
GFP3 15 Frag1 (SEQ ID NO: 20)
AGACGGGCGACAGATaggccataaattgggtgctgaagtctttgcgagagattgtcttgattttggttgggaagatttagcttc
tacaattaataacatgatgtatcttgatataccgtttattgcttttactgcaaataacgataattgggtcaagcaagatgaagt
tatcacattgttatcaaatattcgtagtaatcgatgcaagatatattctttgttaggaagttcgcatgacttgagtgaaaattt
agtggtcctgcgcaatttttatcaatcggttacgaaagccgctatcgcgatggataatgatcatctggatattgatgttgatat
tactgaaccgtcatttgaacatttaactattgcgacagtcaatgaacgccgaatgagaattgagattgaaaatcaagcaatttc
tctgtcttaaTGCGGGCAGGTTATGTAGGTTGAGAGATCGttgacggctagctcagtcctaggtacagtgctagctactagtga
aagaggagaaatactagATGTCGAAGGGCGAAGAGCTTTTCACAGGGGTAGTACCAATTTTAGTAGAGTTGGATGGTGATGTAA
ATGGTCATAAGTTTTCAGTACGTGGGGAAGGTGAAGGGGACGCGACAAATGGTAAATTGACTCTTAAATTTATCTGCACTACCG
GCAAATTAC
GFP3 15 Frag2 (SEQ ID NO: 21)
CTACCGGCAAATTACCCGTTCCATGGCCTACTCTGGTGACGACACTGACCTACGGTGTTCAGTGTTTCAGTCGTTACCCTGACC
ATATGAAACGCCATGATTTCTTCAAGTCGGCTATGCCCGAAGGCTACGTTCAGGAACGTACAATTTCTTTCAAGGATGACGGGA
CGTACAAGACTCGTGCTGAAGTAAAGTTTGAAGGGGATACATTGGTAAATCGTATTGAGTTGAAGGGTATTGACTTTAAGGAGG
ATGGAAACATCTTAGGCCATAAGCTTGAATACAACTTCAACTCCCACAACGTTTATATCACAGCTGACAAACAGAAAAACGGAA
TTAAAGCTAACTTTAAGATCCGCCACAATGTTGAAGACGGAAGCGTTCAATTAGCGGATCACTACCAGCAGAACACACCCATTG
GGGATGGACCTGTCTTGTTGCCAGATAATCATTATCTGAGTACACAATCCGTGTTATCAAAGGACCCGAATGAAAAGCGTGACC
ACATGGTGCTGCTTGAGTTTGTCACAGCGGCAGGGATCACGCACGGCATGGACGAACTGTACAAGTAAaacggcccggagggtg
gcgggcaggacgcccgccataaactgccaggcatcaaattaagcagaagg
GFP3 15 Frag3 (SEQ ID NO: 22)
aaattaagcagaaggccatcctgacggatggcctttttgcgtttGCACCAGTGATCAGGGTTATTCCCTTGGGAaatctattga
gatattctatcactcaaatagcaatataaggactctctgaattcatgaaatttggaaactttttacttacataccaacctcccc
aattttctcaaacagaggtaatggaacgtttggttaaattaggtcgcatctctgaggagtgtggttttgataccgtatggttac
tggagcatcatttcacggagtttggtctacttggtaacccttatgtcgctgctgcatatttacttggcgcgactaaaaaattga
atgtaggaaccgccgctattgttcttcccacagcccatccagtacgccaacttgaagatgtgaatttattggatcaaatgtcaa
aaggacgatttcggtttggtatttgccgagggctttacaacaaggactttcgcgtattcggcgcggatatgaataacagtcgcg
ccttagcggaatgctggtacgggctgataaagaatggcatgacagagggatatatggaagctgataatgaacatatcaagttcc
ataaggtaaaagtaaaccccgcggcgtatagcagaggtggcgcaccggtttatgtggtggctgaatcagctgcgacgactgagt
gggcagctcaatttggcctaATCAGTTCTGGACCA
pUCIDT-AMP (SEQ ID NO: 6)-same plasmid as SEQ ID NO: 6
ATCAGTTCTGGACCAGCGAGCTGTGCTGCGACTCGTGGCGTAATCATGGTCATAGCTGTTTCCTGTGTGAAATTGTTATCCGCT
CACAATTCCACACAACATACGAGCCGGAAGCATAAAGTGTAAAGCCTGGGGTGCCTAATGAGTGAGCTAACTCACATTAATTGC
GTTGCGCTCACTGCCCGCTTTCCAGTCGGGAAACCTGTCGTGCCAGCTGCATTAATGAATCGGCCAACGCGCGGGGAGAGGCGG
TTTGCGTATTGGGCGCTCTTCCGCTTCCTCGCTCACTGACTCGCTGCGCTCGGTCGTTCGGCTGCGGCGAGCGGTATCAGCTCA
CTCAAAGGCGGTAATACGGTTATCCACAGAATCAGGGGATAACGCAGGAAAGAACATGTGAGCAAAAGGCCAGCAAAAGGCCAG
GAACCGTAAAAAGGCCGCGTTGCTGGCGTTTTTCCATAGGCTCCGCCCCCCTGACGAGCATCACAAAAATCGACGCTCAAGTCA
GAGGTGGCGAAACCCGACAGGACTATAAAGATACCAGGCGTTTCCCCCTGGAAGCTCCCTCGTGCGCTCTCCTGTTCCGACCCT
GTCGCTTACCGGATACCTGTCCGCCTTTCTCCCTTCGGGAAGCGTGGCGCTTTCTCATAGCTCACGCTGTAGGTATCTCAGTTC
GGTGTAGGTCGTTCGCTCCAAGCTGGGCTGTGTGCACGAACCCCCCGTTCAGCCCGACCGCTGCGCCTTATCCGGTAACTATCG
TCTTGAGTCCAACCCGGTAAGACACGACTTATCGCCACTGGCAGCAGCCACTGGTAACAGGATTAGCAGAGCGAGGTATGTAGG
CGGTGCTACAGAGTTCTTGAAGTGGTGGCCTAACTACGGCTACACTAGAAGAACAGTATTTGGTATCTGCGCTCTGCTGAAGCC
AGTTACCTTCGGAAAAAGAGTTGGTAGCTCTTGATCCGGCAAACAAACCACCGCTGGTAGCGGTGGTTTTTTTGTTTGCAAGCA
GCAGATTACGCGCAGAAAAAAAGGATCTCAAGAAGATCCTTTGATCTTTTCTACGGGGTCTGACGCTCAGTGGAACGAAAACTC
ACGTTAAGGGATTTTGGTCATGAGATTATCAAAAAGGATCTTCACCTAGATCCTTTTAAATTAAAAATGAAGTTTTAAATCAAT
CTAAAGTATATATGAGTAAACTTGGTCTGACAGTTACCAATGCTTAATCAGTGAGGCACCTATCTCAGCGATCTGTCTATTTCG
TTCATCCATAGTTGCCTGACTCCCCGTCGTGTAGATAACTACGATACGGGAGGGCTTACCATCTGGCCCCAGTGCTGCAATGAT
ACCGCGAGACCCACGCTCACCGGCTCCAGATTTATCAGCAATAAACCAGCCAGCCGGAAGGGCCGAGCGCAGAAGTGGTCCTGC
AACTTTATCCGCCTCCATCCAGTCTATTAATTGTTGCCGGGAAGCTAGAGTAAGTAGTTCGCCAGTTAATAGTTTGCGCAACGT
TGTTGCCATTGCTACAGGCATCGTGGTGTCACGCTCGTCGTTTGGTATGGCTTCATTCAGCTCCGGTTCCCAACGATCAAGGCG
AGTTACATGATCCCCCATGTTGTGCAAAAAAGCGGTTAGCTCCTTCGGTCCTCCGATCGTTGTCAGAAGTAAGTTGGCCGCAGT
GTTATCACTCATGGTTATGGCAGCACTGCATAATTCTCTTACTGTCATGCCATCCGTAAGATGCTTTTCTGTGACTGGTGAGTA
CTCAACCAAGTCATTCTGAGAATAGTGTATGCGGCGACCGAGTTGCTCTTGCCCGGCGTCAATACGGGATAATACCGCGCCACA
TAGCAGAACTTTAAAAGTGCTCATCATTGGAAAACGTTCTTCGGGGCGAAAACTCTCAAGGATCTTACCGCTGTTGAGATCCAG
TTCGATGTAACCCACTCGTGCACCCAACTGATCTTCAGCATCTTTTACTTTCACCAGCGTTTCTGGGTGAGCAAAAACAGGAAG
GCAAAATGCCGCAAAAAAGGGAATAAGGGCGACACGGAAATGTTGAATACTCATACTCTACCTTTTTCAATATTATTGAAGCAT
TTATCAGGGTTATTGTCTCATGAGCGGATACATATTTGAATGTATTTAGAAAAATAAACAAATAGGGGTTCCGCGCACATTTCC
CCGAAAAGTGCCACCTGACGTCTAAGAAACCATTATTATCATGACATTAACCTATAAAAATAGGCGTATCACGAGGCCCTTTCG
TCTCGCGCGTTTCGGTGATGACGGTGAAAACCTCTGACACATGCAGCTCCCGGAGACGGTCACAGCTTGTCTGTAAGCGGATGC
CGGGAGCAGACAAGCCCGTCAGGGCGCGTCAGCGGGTGTTGGCGGGTGTCGGGGCTGGCTTAACTATGCGGCATCAGAGCAGAT
TGTACTGAGAGTGCACCAAATGCGGTGTGAAATACCGCACAGATGCGTAAGGAGAAAATACCGCATCAGGCGCCATTCGCCATT
CAGGCTGCGCAACTGTTGGGAAGGGCGATCGGTGCGGGCCTCATCGCTATTACGCCAGCTGGCGAAAGGGGGATGTGCTGCAAG
GCGATTAAGTTGGGTAACGCCAGGGTTTTCCCAGTCACGACGTTGTAAAACGACGGCCAGTGCAACGCGATGACGATGGATAGC
GATTCATCGATGAGCTGACCCGATCGCCGCCGCCGGAGGGTTGCGTTTGAGACGGGCGACAGAT

Reaction mix compositions were setup as previously described in Example 3. The plurality of distinct dsDNA fragments and reaction mix were incubated as previously described in Example 2 and transformed as previously described in Example 1.

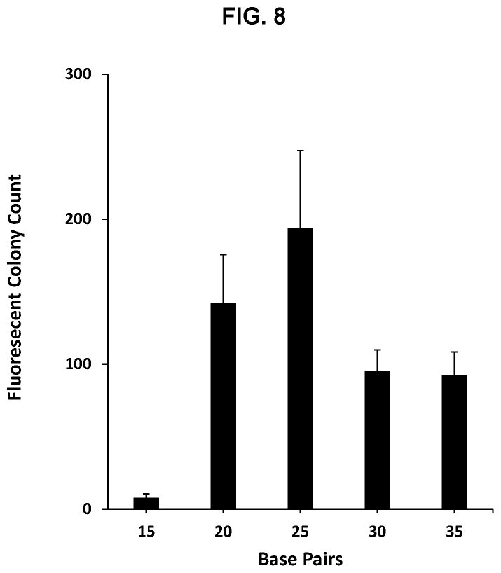

FIG. 8 shows that different overhang lengths can be used in the assembly reaction.

1. A method for the assembly of a plurality of double stranded DNA (dsDNA) fragments into a covalently bound circular dsDNA molecule, the method comprising:
(a) combining a plurality of distinct dsDNA fragments with a reaction mixture comprising an exonuclease and a recombinase to form a DNA reaction mixture;
wherein each individual dsDNA fragment comprises one or more terminal single-stranded nucleotides that are complementary to terminal single-stranded nucleotides of an independent dsDNA fragment;
(b) subjecting the DNA reaction mixture to a hybridization incubation to form a hybridized DNA reaction mixture;
(c) subjecting the hybridized DNA reaction mixture to a deactivation incubation to form a deactivated DNA reaction mixture;
(d) transforming the deactivated DNA reaction mixture into a competent host cell; and
(e) incubating the transformed competent host cell under conditions sufficient to assemble and replicate one or more covalently bound circular dsDNA molecules comprising the plurality of distinct dsDNA fragments.

2. The method of claim 1, wherein the recombinase is selected from Uvsx from a bacteriophage, Rad51 or Dmc1 from a eukaryote, RadA from archaea, or RecA from E. coli.

3. The method of claim 2, wherein the recombinase is RecA from E. coli.

4. The method of claim 1, wherein the reaction mixture further comprises ATP.

5. The method of claim 1, wherein the exonuclease is T5 Exonuclease.

6. The method of claim 1, wherein the reaction mixture further comprises a DNA polymerase and a ligase.

7. The method of claim 1, wherein the competent host cell is an E. coli cell.

8. The method of claim 1, wherein the one or more terminal single-stranded nucleotides that are complementary overlap with terminal single-stranded nucleotides of the independent dsDNA fragment by about 10 nucleotides to about 120 nucleotides.

9. The method of claim 8, wherein the one or more terminal single-stranded nucleotides that are complementary overlap with terminal single-stranded nucleotides of the independent dsDNA fragment by about 20 nucleotides to about 60 nucleotides.

10. The method of claim 9, wherein the one or more terminal single-stranded nucleotides that are complementary overlap with terminal single-stranded nucleotides of the independent dsDNA fragment by about 20 nucleotides to about 35 nucleotides.

11. The method of claim 10, wherein the one or more terminal single-stranded nucleotides that are complementary overlap with terminal single-stranded nucleotides of the independent dsDNA fragment by about 25 nucleotides to about 30 nucleotides.

12. The method of claim 1, wherein the hybridization incubation comprises a hybridization temperature of about 25° C. to about 50° C. for about 5 minutes to about 120 minutes.

13. The method of claim 12, wherein the hybridization incubation comprises a hybridization temperature of about 35° C. to about 45° C. for about 10 minutes to about 20 minutes.

14. The method of claim 13, wherein the hybridization incubation comprises a hybridization temperature of about 42° C. for about 20 minutes.

15. The method of claim 1, wherein the deactivation incubation comprises a deactivation temperature of about 60° C. to about 70° C. for about 5 minutes to about 120 minutes.

16. The method of claim 15, wherein the deactivation incubation comprises a deactivation temperature of about 65° C. for about 20 minutes.

17. The method of claim 1, wherein the deactivation incubation comprises a deactivation temperature of less than about 5° C. for about 20 minutes.

18. The method of claim 1, wherein the reaction mixture further comprises one or more crowding agents, one or more chaperone agents, or a combination thereof.

19. The method of claim 18, wherein the one or more crowding agents comprises polyethylene glycol (PEG).

20. The method of claim 18, wherein the one or more chaperone agents comprises a diol or a polyol selected from substituted straight or branched alkylene glycols, pentaerythritol, sorbitol, diethylene glycol, dipropylene glycol, neopentyl glycol, propylene glycol and ethylene glycol ethers, 1,2-ethylene glycol, 1,2-PrD, 1,3-PrD, 1,4-butanediol, 1,5-pentanediol, 1,6-hexanediol, 2-methyl-1,3-propanediol, 2,2′-dimethylpropylene glycol, 1,3-butylethylpropanediol, methyl propanediol, methyl pentanediols, propylene glycol methyl ether, propylene glycol ethyl ether, propylene glycol butyl ether, diethylene glycol phenyl ether, propylene glycol phenol ether, propylene glycol methyl ether, tri-propylene glycol methyl ether, propylene glycol isobutyl ether, ethylene glycol methyl ether, or combinations thereof.

21. The method of claim 1, further comprising isolating the covalently bound circular dsDNA molecules comprising the plurality of distinct dsDNA fragments from one or more competent host cells.

22. The method of claim 21, further comprising sequencing the covalently bound circular DNA molecule comprising the plurality of distinct dsDNA fragments following isolation from the competent host cell.

23-32. (canceled)
